SEQUENCE LISTING (45 sequences)

SEQ ID NO: 1 - DNA, 51 bp, Artificial Sequence
  adaptor (universal genome walker)
gtaatacgactcactatagggcacgcgtggtggtcgacggcccgggctggt

SEQ ID NO: 2 - DNA, 8 bp, Artificial Sequence
  adaptor (universal genome walker)
accagccc

SEQ ID NO: 3 - DNA, 22 bp, Artificial Sequence
  primer
gtaatacgactcactatagggc

SEQ ID NO: 4 - DNA, 19 bp, Artificial Sequence
  primer
actatagggcacgcgtggt

SEQ ID NO: 5 - DNA, 26 bp, Artificial Sequence
  primer
aatttgctccaatatcttagctctac

SEQ ID NO: 6 - DNA, 26 bp, Artificial Sequence
  primer
agacagccatttctttttgtagatac

SEQ ID NO: 7 - DNA, 24 bp, Artificial Sequence
  primer
agcggataacaatttcacacagga

SEQ ID NO: 8 - DNA, 32 bp, Artificial Sequence
  primer
tagacggatccttctttttgtagatacaagat

SEQ ID NO: 9 - DNA, 22 bp, Artificial Sequence
  primer
gatccattattagagattgagc

SEQ ID NO: 10 - DNA, 22 bp, Artificial Sequence
  primer
catggctcaatctctaataatg

SEQ ID NO: 11 - DNA, 28 bp, Artificial Sequence
  primer
gggctggaaagcttaagagaaattggta

SEQ ID NO: 12 - DNA, 30 bp, Artificial Sequence
  primer
ggggttttgtttttggatcctgggtgtgtt

SEQ ID NO: 13 - DNA, 27 bp, Artificial Sequence
  primer
ccatcctaatacgactcactatagggc

SEQ ID NO: 14 - DNA, 28 bp, Artificial Sequence
  primer
gggcaggtttctagaattcagcggccgc

SEQ ID NO: 15 - DNA, 30 bp, Artificial Sequence
  primer
gtgaaactcgacccgttccttaaaaacttc

SEQ ID NO: 16 - DNA, 32 bp, Artificial Sequence
  primer
gctttccaatgagagccatggttttaaacctt

SEQ ID NO: 17 - DNA, 27 bp, Artificial Sequence
  primer
tattaccttcactggatctcttccctc

SEQ ID NO: 18 - DNA, 28 bp, Artificial Sequence
  primer
gccttaagctttgttgatcatccacatc

SEQ ID NO: 19 - DNA, 23 bp, Artificial Sequence
  primer
gtttgcattgtttccatgggaaa

SEQ ID NO: 20 - DNA, 24 bp, Artificial Sequence
  primer
agcggataacaatttcacacagga

SEQ ID NO: 21 - DNA, 16 bp, Artificial Sequence
  primer
aagctttttttttttg

SEQ ID NO: 22 - DNA, 16 bp, Artificial Sequence
  primer
aagctttttttttttc

SEQ ID NO: 23 - DNA, 16 bp, Artificial Sequence
  primer
aagcttttttttttta

SEQ ID NO: 24 - DNA, 13 bp, Artificial Sequence
  primer
aagcttgattgcc

SEQ ID NO: 25 - DNA, 13 bp, Artificial Sequence
  primer
aagcttcgactgt

SEQ ID NO: 26 - DNA, 13 bp, Artificial Sequence
  primer
aagctttggtcag

SEQ ID NO: 27 - DNA, 13 bp, Artificial Sequence
  primer
aagcttctcaacg

SEQ ID NO: 28 - DNA, 13 bp, Artificial Sequence
  primer
aagcttagtaggc

SEQ ID NO: 29 - DNA, 13 bp, Artificial Sequence
  primer
aagcttgcaccat

SEQ ID NO: 30 - DNA, 13 bp, Artificial Sequence
  primer
aagcttaacgagg

SEQ ID NO: 31 - DNA, 13 bp, Artificial Sequence
  primer
aagcttttaccgc

SEQ ID NO: 32 - DNA, 28 bp, Artificial Sequence
  primer
ttctaggcgaaaaccaagtgggcctaat

SEQ ID NO: 33 - DNA, 27 bp, Artificial Sequence
  primer
cccacactgaccccaacaaacaatagc

SEQ ID NO: 34 - DNA, 24 bp, Artificial Sequence
  primer
aggccatggtcggtgccgggaaaa

SEQ ID NO: 35 - DNA, 24 bp, Artificial Sequence
  primer
agcggataacaatttcacacagga

SEQ ID NO: 36 - DNA, 26 bp, Artificial Sequence
  primer
gacagtatagttcatggcttggttgg

SEQ ID NO: 37 - DNA, 27 bp, Artificial Sequence
  primer
aggttcttttaatcaggcaatcttctt

SEQ ID NO: 38 - DNA, 30 bp, Artificial Sequence
  primer
gcgggatcctatttttgtgaattggaaatg

SEQ ID NO: 39 - DNA, 24 bp, Artificial Sequence
  primer
cgccagggttttcccagtcacgac

SEQ ID NO: 40 - DNA, 1499 bp, Artificial Sequence
  promoter
agcggataacaatttcacacaggaaacagctatgaccatgattacgccaagcttggtaccgagctcggatccactagtaacggccgccagtgtgctggaattcggcttactatagggcacgcgtggtcgacggcccgggctggtaactttaagagaaattggtaaaattcctagagagaattgtaattaatataggagaatgattttaattctaatgttgtatccattttcgataaagttaaataaagtgtcgtagacgaccatcattcttaatccatttgtacttatcaaatttgtatctgagatttaagttcaaattcacactaaaacaatcgaaatgtatgcgacaatcacaatggaaaatacgtatgatgtattccatcacctttcaagttctaacctaggatatgttttggaatatttgagatttattaaattattcttttatccgttgacagtttattttttgtttaacgatgtatgtaagaaacgacgaaatatgtgattaaaccaagatcgcatacaaataagagctagatcctaaagatatataaaagtatgatcaacaacgtacaaaacgtttcttttcgatgataattatcttaagaacttcaaggttaatttagatctcttaattaaaaaatttcatagataatgcatccgtgaacaagaaaaaacataaagaacccatggttgtcctaatttttgtagtaaataagcgtagttcaagacacaagtaagaatgacgttaccacatgttaatctagattccaaaacttgagcttgagagcacgttacgaaaataatctacgaaaacgagtaagtcgtctaagttcgttttcgtttatttgacacgtaagatactcgtattgaaagaagacgaaaaatggaaaaaagtaaagaaggtaaggaggtgggtgagtccaaaggaaacataccaaattcatgcaagaactatgagattcagaaattaagagaaaagtgtggaaatcatgtaactaaatttaaaatacatataggtactattttctttccttttctattgaaacaaagagaccaagggggaattagggtatatggcattggcagacataaaaataataaagttaaatcaaattgggtcccaaactcaccaaagaggaaattcagtgttgaataaagccaattagccaaagccaaagccaaagccacctcctctctttcccacatacatgcatgaaatttcatgggcccattctttttatcatcacatttttaataattttatcttcttcttcttcttcttcttcttcttcttcttcttcttcttcttcttcttctttttttaatcaatttcttcccactttccaatcctaaataaatttcactataaataccccttcattataacttgatccaacacacccaccaaccaaaaacaaaaccttgataccaaagagttcttttttctttatttgcacaaaccaaatcttgtatctacaaaaagaaatggctgtcta

SEQ ID NO: 41 - DNA, 1319 bp, Artificial Sequence
  promoter
aggaaacagctatgaccatgattacgccaagcttaagagaaattggtaaaattcctagagagaattgtaattaatataggagaatgattttaattctaatgttgtatccattttcgataaagttaaataaagtgtcgtagacgaccatcattcttaatccatttgtacttatcaaatttgtatctgagatttaagttcaaattcacactaaaacaatcgaaatgtatgcgacaatcacaatggaaaatacgtatgatgtattccatcacctttcaagttctaacctaggatatgttttggaatatttgagatttattaaattattcttttatccgttgacagtttattttttgtttaacgatgtatgtaagaaacgacgaaatatgtgattaaaccaagatcgcatacaaataagagctagatcctaaagatatataaaagtatgatcaacaacgtacaaaacgtttcttttcgatgataattatcttaagaacttcaaggttaatttagatctcttaattaaaaaatttcatagataatgcatccgtgaacaagaaaaaacataaagaacccatggttgtcctaatttttgtagtaaataagcgtagttcaagacacaagtaagaatgacgttaccacatgttaatctagattccaaaacttgagcttgagagcacgttacgaaaataatctacgaaaacgagtaagtcgtctaagttcgttttcgtttatttgacacgtaagatactcgtattgaaagaagacgaaaaatggaaaaaagtaaagaaggtaaggaggtgggtgagtccaaaggaaacataccaaattcatgcaagaactatgagattcagaaattaagagaaaagtgtggaaatcatgtaactaaatttaaaatacatataggtactattttctttccttttctattgaaasraagagannaagggggaattagngtatatggcattggcagacataaaaataataaagttaaatcaaattgggtcccaaactcaccaaagaggaaattcagtgttgaataaagccaattagccaaagccaaagccaaagccacctcctctctttcccacatacatgcatgaaatttcatgggcccattctttttatcatcacatttttaataattttatcttcttcttcttcttcttcttcttcttcttcttcttcttcttcttcttcttctttttttaatcaatttcttcccactttccaatcctaaataaatttcactataaataccccttcattataacttgatccaacacacccaggatccattattagagattgagccatgg

SEQ ID NO: 42 - DNA, 1735 bp, Artificial Sequence
  promoter
agcggataacaatttcacacaggaaacagctatgaccatgattacgccaagcttggtaccgagctcggatccactagtaacggccgccagtgtgctggaattcggcttgtaatacgactcactatagggcacgcgtggtccacggcccgggctggtaactagaagctaaaggacgacgtcaacataattaaaattactccaagataattaaaattaaaaatatcttatattttatggcgttacatcttcctttcctcttcttcttttttctgctgcgatttcttcccatctatttcttcttttactcttatttttttctttacattgtttagatttgggtaaccaaatctgatttctttctatcgtctttcttctttttctctttttttttccgctgcgatttcttcccattgtctatcgttttttcctctttttttttttacatcgtaaccaaatctaaaagatcgtatataaagaatcttcaaaaaaaaaaattgtttagattggagtagccaaatttaaacaatcgcgtaaaaaaaataaacgatcgtagacaaatctaaacgatcgtgcacaaaaagatttaaaaaaatcgtttagtcaaatctaaacaattgtataaccaaattaaacgatagaattgaaataataaatcggttagatttggctatccaaatttaaatgaccaaatctaaacgatcgtataccaaatctaaacgatcgkataccaaatctaaatgatcatgtaccaaatatattatgcacattgttggcagggtggttgacggaacattttgtatattttctattatgggtttgtagaattttttcattttcgaaattgttctatacaatataaatataaatattttaccacttcgttatattttcgaaaagaccccttaaataaattgaattcgcatataattaaaattttttcccaaaaaaagtagactatgtctatctaaaaatttgattcccaatatagaacaaattctcaaaatgaacaaacatttgaaattctcgatatagaaaacatttacttattttgaattgggacatattccaaagtttattccaaacgtaactttgaaggaaaagttgattgagattacatccatatttttgtttttcatattgaatttcatggaaaattaaaatgcacacaaaatgatgtatgagattaaaccaaagtttatcgttattgaattcttttattaaaaaaccaacaaaattttaaaacttgtttgcaatagaccaatatagttaatccatcgtggtctattgtagataaattgtaatattttgttatatttaataaatattttgatttattttgatatatttgtatttagataacaaaattaagatttaaatattattttatatcttaatataaacatttgttaattttttctattttagaccatttctcttatttttatataacattttaataactaaatgatgtgacacacactaatattatttttatccaaagaaaataatgctataaaatatgggtcttctttatcaccttcatgataattatgaaaaataaaataaaatttaattatataattcatttcatctaatcgtacaagctagatattactatatcaacaactttgtgtataaaaagggcaagaaattaagcattatcgtgtgagccactttttctatatctagagatagaaggtttaaaatcatgtctctaattggaaagcttgtgagt

SEQ ID NO: 43 - DNA, 2184 bp, Artificial Sequence
  promoter
ttgtgtggaattgtgagcggataacaatttcacacaggaaacagctatgaccatgattacgccaagcttggtaccgagctcggatccactagtaacggccgccagtgtgctggaattcggcttactatagggcacgcgtggtcgacggcccgggctggtccaatcaccgaacatcatgttatgtaggtgtcgggagatgctacctatctgctgatgttggtttctttctttgaaagatactctcctgactttttagttgttgcatctagagatgtcctctattattttgacaccttttcttctgacggtgtagagcaacacaaaaaaatcttgaatttctattaatggaatgagctatatctatacaaattggaaccatatgacaaattaagaagattcctttctgaatattatgcaatagaaatactcaccaggtgtaatgatgcaccttatagagaaaacttcgacgaacaagagacggctactaagttttagtagaatgggtatttctgacctactatgtttcaggatgcgaggatcttcatggtcaattgtgaccgatggtggagaactgaaaatatttcccatctcaatgaaatactaaaacaacatatcttagaggttgaactatttgatatctagggaatacattttatgggaccgttttctagttgttccggcaaacacgcattcgagacgggacgttcatgtcgcataccacggaggatccgcatgtaagctatccaagcaatacttctaccctttttgtcttctttaataatatattttttacttactaagatagtttctaaatttgttgtagaatcgaatgctggaacttcagtcctagcttacctcagtgggtacttagccactctctggggacaagatatgcgagatggcgttggatagacgattggactactcaaaaggccttggttggggacctaagtctagggcccacaagacggccagtgtgagtagttccacgacctcatgtttgtagtccacggtagagctccaattatggactaagcttgatcaagctgtgcaacggattgaagaacaaacaagaaatcacgatgcgttagcttcaaaagtggaatgaatgtgaaagttcatagaagacatgagtcgggcacagtaaggaccacaacattatctttagctttgcgatacgtatanntatttccattattcttaagtttttgaattacagtattcagtgatgatatgcatatatatgtaccaaacgtagccacttttgtataattgtaggacctgtggtgtagaatggcatatgaggctcgttaaaagacatacgattttctttgtgctttttttaacgaggaatattttttatttgtattatgaactttattacatttcttgaatttctttgtattatgaagatttaattttttgttgaatttttgtttgtattttgtaatttactaatttattttaaattttctttaattgaatcgataacgaatgcaaatattttacgaaaaaaacttataggaaaatatttcaaaaaaataaaaaattacatatttaaaatatttttcgacgcattacatatgtggaaaatatggtgcaaacatcacatcggggatggttattaccgacgcatgaatgacaccgaatatataaacgtaaggaatagttattcctgacgcataactgctgtcggaactgtggaagttagttctcgacattattaacacttacgtcgacgtttttatgcatcgggagtcgctccacttcttgtagtgaagaaattttgcctataatgtcggtttaaaaccgacattaaaggccaaatttcttctagtgcataatcaatatmcaaaagttcaattccaaaaattacatttctctagaaattccgtgtgaacaattgtcataaaggttttaagtgaattgaaaatttcaaaacgtaattggattaagcgagaaaattattttaatcaccattcaaaagttattaacaatgaaaaatatggaagataagatttcaaaattacgtaatttacttctacgtttctttctttcccctttagtaacttcactcatatctttatatacgttccatcccttcacattctcatacaaaattctctttcaatatcaactctcctctcttaactcacccttttttcaaatggaaacaatgcaaac

SEQ ID NO: 44 - DNA, 985 bp, Artificial Sequence
  promoter
tggaattgtgagcggataacaatttcacacaggaaacagctatgaccatgattacgccaagcttggtaccgagctcggatccactagtaacggccgccagtgtgctggaattcggcttactatagggcacgcgtggtcgacggcccgggctggtaaattttgaaaagttaggagatattttttacatataagagatattttttataatgtaacattttttttactagacggttgagtcgagttaggttaaagaaaggaaaactataaaataatttttaattattaaatacataaacaatactttgtattctatattaattaaaatgactattgaattgttaagatgtaggtatctaaggacaagaagtctcgagttcaaatcttcaacctcaaaatatactgcaagatagtaactaatgaattatatttgactaaatcatgtagcaaaagaaaatcaaatttatcatgttaaatatggtcaagccggagcattaacaacaacaattcatatttgtggttgatagtacttgactagaatttagagagtacttgactagaataaaaattgggggacccactacgacgtcagcttgccttgcttagcaattaagctatcacctcttagtctatagcttcgtgcgctgcattaaacggtattctcacacttttcttttctttttaccgcacccgtccggttaatggctcccccactttttaccttccccgaatccacgccagttgccaacatgcgaagcagcaagtacaatattgtcattttgcattaaccaaaatgacacgtcggatgtcatttatgtaattaagctacaaagccacggttagtttccgaacccccacgatccagtacttacgtgtctcctataaatcttagaagcaacgtctttaccggaatcaactcattaggtatcccattttcatctatcaattcacccttgaaactgcttttcccggcaccgactatggcctc

SEQ ID NO: 45 - DNA, 2455 bp, Artificial Sequence
  promoter
aagcttgcatgcctgcaggtcgactctagatcaatcaaacatttatttaaatagaacgttttagtggtttttggattttatttatcttttttattatttacattcaatttagatttaactcttgcagaagatggaggaagagaatttttagaaattgaactgaaatagacttaattattaaaaatcaaaagaaaaatggtgccaacaaaggtgactaagagtgtaatgaattggaattagaactttccttctgtatagatataattgatgttttccttaactttatttttatggtggttatttattaataactgaatttttaagagttcttttaataaccaaatgttataggattcaattgattgttttatgagattaggcaaacactttatattggagaaataatttagtgtagaaagtaattttcattttggattgtttagatgaacatcaaatcttgcaacaacattcagttaagtatatataaatatatagagccaccaacctcaaatacaatatcttcggaagcaaaatattatacataatatggaaagaagagtagtactggtacatgaatcttacgaagaatttaagtattattggcttttccaatgcagaagtctcaacaaatcacattttaaaaaccgattgaataaacatgcaagtaagacttttgaaaaaacaagcattcaaacctcatatcaattatctctatatgcaaaatgttaggtcaaatgagtaatgaaattaaggacaaatcaactaaaaagaatcaataaagtgaatcgaaaagaaacaaatatcaatcaaacctaatgtatacgtgattgatgatgcagtgtgttttgagatatggacattttgataaacaacaaacctccactccaatacgagaagagaggcattgagtgacagattagtgccctattgaagagggtaagtccaaaacaaaacaaacacaaaacatggtgaagaaatgttatgaataaatggcagggaaagacatggttgtacatgtggtgtgagttttcttctttcaaatctgtgaataaattggattacgacccaacaagagaaacactgtttggaaaccatgacagggctaccccatggcgtgaatatcaagtatttaattaattaagctctcatccccgccattcgtttttttattcgattcatatcttatattttatatacgaataattcttgagtttgatttcaatttagttcgtcaatagtaatattttaaactatgttaatatataaaaagtaaatgcgaatgattctattagtattcactttatatcactccttcttagagtaaagtttttaaagtgggaagggaaatggaatacgacgtgtgattggtagttaaatttccttatcgacgaggttactgtttccttacttatatatatggagtcatcctcaatttttcaactctcaacttccaattatacaagcaaaacattcaataccatacatgcatctttttagaaagaaaagaagttctctcttggactttttttttcaattcaactatgcacctttgttattttagttttataatttttgttgtttcttccgtttaatcaagttgttgtaatcaactttattgattcaaacacatagatattttgtttaatagtatcagtatataatagggttagaataacctttcaaatatgtgtttttaaaaaatcaaatcactttaaaaattaaaatatgtttaattagtgtatgtttttctttttaagtattaaaccacgataaaaagtgctttaacacttataaaaaattagattaatttaaaggaagttgtctgaacggcaaaattgacaaaatataacaaagtttaatgaacattgttcgaaatgtttcgaagaggaaagaaaacattaagtttgaaatatctcgagttaaatacatatcatcccatagtaatatataacaaaacaaacttaaatctgaaaaaaaaattgatgttaataagaaaaagagatcaaactcttaattttttaaaaaaataatggtgaaaaaaactgaaaattttccaatattgtttaatttcaaattgatccaaaaattaaagttaaaaaagcattaaacaaaacaattcaaaacctagctactacacatttacgaaaatatatgatacacaaaggatttttgggtgtaaacatcttttttattttatatacaccaaacttcgtatatattcacacataaagaaggaaaaagaattaatgcaagggtgtggccaattacgtaccgtcgtcatatcctactcatccgttacgttctcaaatctctctctctccctgctctcctaattatttctgccagcgaccatatttcattttcaattgtgtgtttaaaaagccgagaatcgcaatcctttttcctccactcttaattcatttccaattcacaaaaataggatccgccaccatgg

(19) United States
(12) Patent Application Publication
(10) Pub. No.: US 2002/0133850 A1
(43) Pub. Date: Sep. 19, 2002

(54) Melon promoters for expression of transgenes in plants

(76) Inventors: Stephanie K. Clendennen, Portland, OR (US); Jill A. Kellogg, Bend, OR (US)

Correspondence Address: PERKINS COIE LLP, P.O. BOX 2168, MENLO PARK, CA, 94026, US

(21) Appl. No.: 09/811,093

(22) Filed: Mar. 16, 2001

Related U.S. Application Data
(60) Provisional application No. 60/190,414, filed on Mar. 17, 2000.

(51) Int. Cl. C12N 15/82; C12N 15/29; A01H 5/00

(52) U.S. Cl. 800/287; 800/283; 536/23.6

(57) ABSTRACT

The present invention is directed to melon promoters capable of promoting the expression of heterologous genes in transformed melon fruit. The invention provides melon fruit-associated promoters, heterologous nucleic acid constructs, vectors, kits, transformation methods, transgenic plant cells and transgenic plants comprising such promoters.

This application claims priority to U.S. Provisional application Ser. No. 60/190,414, filed on Mar. 17, 2000, expressly incorporated by reference herein.

### FIELD OF THE INVENTION

The present invention relates to melon fruit-associated promoters and heterologous nucleic acid constructs, vectors, kits, and transformation methods employing such promoters. The invention further relates to transgenic plant cells and plants transformed with heterologous nucleic acid constructs comprising such melon fruit-associated promoters.

### REFERENCES

Adams, D. O., and Yang, S. F., Plant Physiology 70:117-123, 1977.

Aggelis et al., J. Experimental Botany 48: 769-778, 1997.

Atkinson, RG et al., Plant Mol Biol 38: 449-460, 1998.

Ausubel, FM, et al., in CURRENT PROTOCOLS IN MOLECULAR BIOLOGY, John Wiley and Sons, Inc., Media, Pa., 1992.

Balague et al, Eur J Biochem 212:1 27-34, 1992.

Becker, et al., Plant Mol Biol 6: 1195-7, 1992.

Bestwick, et al., U.S. Pat. No. 5, 859,330, 1995.

Bhalla PL and Smith N, Molecular Breeding 4: 531-41, 1998.

Blume, B et al., Plant J., 12: 731-746, 1997.

Bouquin, T., et al., Plant Mol Biol 35: 1029-35, 1997.

Brunke, KJ and Wilson, SL, European Patent Publication No. 0 559 603 A2, published Sep. 08, 1993.

Cabrera-Ponce JL et al., Plant Cell Reports 16: 255-260, 1997.

Catlin D et al., Plant Cell Reports 7: 100-103, 1988.

Chang,S. et al., Plant Molecular Biology Reporter, Volume 11 (2): 113-6, 1993.

Clendennen et al., In BIOLOGY AND BIOTECHNOLOGY OF THE PLANT HORMONE ETHYLENE II. Eds: AK Kanellis et al. Kluwer Academic Publishers. pp. 371-380, 1999.

Cordes, S, et al., The Plant Cell 1:1025-1034,1989.

Deikman, J., et al., Plant Physiol. 100, 2013-2017, 1992.

Deikman, J., et al., Plant Mol. Biol. 37, 1001-1011, 1998.

Delbreil B et al., Plant Cell Reports 12:129-132, 1993.

Franks T et al., Molecular Breeding 4:321-33, 1998.

Gold, M., et al. Proc. Nat. Acad. Sci. 52, 292-297, 1964.

Good, X., et al., Plant Mol. Biol. 26, 781-790, 1994.

Hardegger M et al., Molecular Breeding 4: 119-127, 1998.

Heffera-Estrella, L., et al., Reporter genes for plants, in PLANT MOLECULAR BIOLOGY MANUAL, 2nd Ed., S. B. Gelvin and R. A. Schilperoort, eds. Kluwer Academic Publishers, Boston, C2 pp. 1-32, 1994.

Holdworth et al, Nucleic Acids Res. 15: 731-739, 1987.

Houck, CM and Pear, JR, U.S. Pat. No. 4,943,674, issued Jul. 24, 1990.

Hughes, J. A. et al., Nuc. Acid Res. 15, 717, 1987a.

Hughes, J. A. et al. J. Bact. 169, 3625, 1987b.

Hooykaas, P3, and Schilperoot, RA, in TRENDS IN BIOCHEMICAL SCIENCES, International Union of Biochemistry and Elsevier Science Publishers, v. 10(8):307-309, 1985.

Hosoki T et al., J. Japan Soc. Hort. Sci. 60: 71-75, 1991.

Kende, H. Annu. Rev. Plant Physiol. Plant MoL Biol. 44, 283 -307, 1993.

Kramer, M. G., et al, in A. K. Kanellis, et al. (eds.), BIOLOGY AND BIOTECHNOLOGY OF THE PLANT HORMONE ETHYLENE, Kluwer Academic Publishers, Boston, pp. 307-319, 1997.

Lasserre, E., et al., Mol Gen Genet 251: 81-90, 1996.

Lasserre, E., et al., Mol Gen Genet 256: 211-22, 1997.

Leisner, S. M., and Gelvin, S. B., Proc. Natl. Acad. Sci. USA 85(8):2553-2557, 1988.

Liang P. and Pardee AB, Mol Biotechnol., 3:261-7, 1998.

Miguel CM et al, Plant Cell Reports 18: 387-93, 1999.

Mogilner N et al, Mol Plant Microbe Interact 6(5):673-5, 1993.

Nakamura Y et al., Plant Cell Reports 17:435-440.

Ni M. et al., The Plant Journal 7: 661-676, 1995.

Picton S. et al., Plant Physioloay 103(4): 1471-1472, 1993.

Purseglove, J. W. Tropical Crops, Dicotyledons 1, John Wiley and Sons, New York, 1968.

Rogers S, U.S. Pat. No. 5,034,322, issued Jul. 23, 1991.

Salvatore, F. et al. The Biochemistry of Adenosylmethionine, Columbia University Press, New York, 1977.

Sambrook, J, et al., in MOLECULAR CLONING: A LABORATORY MANUAL, Cold Spring Harbor Laboratory Press, Cold Spring Harbor, N.Y., Vol. 2, 1989.

Sarmento GG et al., Plant Cell Tissue and Organ Culture 31: 185-193, 1992.

Scorza R et al., Plant Cell Reports 14: 589-92, 1995.

Tang X et al., Plant Mol Biol:6 1151-64, 1993.

Trulson AJ et al., Theor AppI Genet 73: 11-15, 1986.

Usdin, E. et al. Transmethylation, Elsevier/North Holland Publishing, New York, 1979.

Van Eck JM, et al., Plant Cell Reports 14: 299-304, 1995.

Van Haaren MJJ, et al., Plant Mol. Bio. 21:625-640, 1993.

Verdaguer, B., et al., Plant Mol Biol 31: 1129-1139, 1996.

Xu, R., Goldman, S., et al., Plant Mol. Biol. 31, 1117-1127, 1996.

Zhang HX and Zeevaart JAD, Plant Cell Reports 18: 640-45, 1999.

Zhu, et al., Plant Cell: 10 1681-9, 1995.

### BACKGROUND OF THE INVENTION

All cultivated forms of cantaloupe belong to the highly polymorphic species, Cucumis melo L., that is grown for its sweet edible fruit (Purseglove, 1968). In the United States, the principal fresh market cantaloupe growing regions are California, Arizona and Texas which produce approximately 96,000 acres out of a total annual acreage of more than 113,000 acres (USDA, 1998). Cantaloupe comprises a $2.8 billion retail market in the United States. It has been estimated that postharvest losses, which are largely attributable to the effects of ethylene, can reach 30% throughout the distribution chain.

Genetic modification of plants to manipulate ethylene biosynthesis has been demonstrated to lead to delayed ripening and extended postharvest life in climacteric fruit, which in turn reduces post-harvest losses resulting from produce that is overripe and senescent. A gene derived from E. coli bacteriophage T3 encoding an enzyme capable of degrading S-adenosylmethionine (SAM) has been introduced into the Cucumis melo genome using standard Agrobacterium binary vectors. Production of S-adenosylmethionine hydrolase (SAMase) in fruit has been demonstrated to alter the ethylene biosynthetic pathway and cause a modified fruit ripening phenotype in cantaloupe (Clendennen et al., 1999).

There are many examples of fruit-specific and ripening-associated promoters from plants. 1-aminocyclopropane-1-carboxylate oxidase (ACO) and polygalacturonase (PG) promoters were isolated from apple, and the promoters direct fruit-specific and ripening-associated expression of a reporter gene in transformed tomato (Atkinson et al., 1998). A tomato ACO promoter has been demonstrated to direct ethylene-responsive reporter gene expression in both tomato and tobacco (Blume et al., 1997). Two other well-characterized examples are the promoters associated with the tomato fruit-specific and ethylene responsive genes E8 and E4 (Deikman, et al, 1992; Xu et al. 1996; Deikman et al., 1998). Ethylene responsive elements have been identified in the tomato E8 and E4 promoters, as well as elements governing organ-specificity and elements associated with ripening. The promoter from the ripening-associated E8 gene was used successfully to drive expression of SAMase in tomato, resulting in a decrease in ethylene production in the fruit (Good el al., 1994; Kramer et al, 1997). A novel ethylene responsive hybrid promoter synthesized from elements of the tomato E8 and E4 promoters has been used for the expression of SAMase in cantaloupe (Clendennen et al., 1999).

Transcriptional regulatory sequences or promoters that regulate gene expression in plants are essential elements in the genetic modification of plants. Most promoters are from about 500-1500 bases. Promoters for expressing heterologous genes in plants can be derived from plant DNA, e.g., the cauliflower heat shock protein 80 (hsp80, Brunke and Wilson, 1993; U.S. Pat. No. 5,612,472), or from other sources, for example, plant viruses e.g., the 35S cauliflower mosaic virus promoter, or bacteria which infect plants, e.g., the nopaline synthase (nos) promoter (Rogers, 1991), the octopine synthase (ocs) promoter (Leisner et al., 1988) and the mannopine synthase (mas) promoter from Agrobacterium. Numerous examples of promoters useful for the expression of heterologous genes in plants are now available (Zhu, et al., 1995; Ni, et al., 1995).

Expression of complete genes or selected sequences of heterologous genes in transgenic plants has often involved the use of constitutive promoters, which drive the expression of a product throughout the plant at all times and in most tissues (e.g., hsp80), the tomato ubiquitin promoter (Picton, et al, 1993), and the raspberry E4 promoter (U.S. Pat. Nos. 5,783,393 and 5,783,394).

Although a limited number of inducible and/or tissue specific promoters are known, it has been demonstrated that nucleic acid sequences placed under the regulatory control of the 5′ non-coding region of the tomato 2AII gene are preferentially transcribed in developing fruit tissue. (See, e.g., Van Haaren, 1993. ) Promoters that provide fruit-specific expression include the E4 and E8 promoter from tomato (Cordes et al., 1989; Bestwick et al., 1995; U.S. Pat. No. 5,859,330) and the tomato 2AII gene promoter. Fruit specific regulation of the kiwifruit actinidin promoter has been reported to be conserved in transgenic petunia plants (Lin et al., 1993).

In the genetic modification of plants it is often important to control the timing and/or tissue specificity of gene expression. Accordingly, there remains a need for identification and characterization of plant promoters that are inducible and/or tissue or plant specific and which are capable of providing for preferential or specific expression of heterologous genes in plants.

### SUMMARY OF THE INVENTION

The present invention provides upstream regulatory regions (promoter sequences) from a number of genes in melon that are expressed primarily or exclusively in fruit including cmACO1, cmACO1/TE4, MEL7, MEL2, cm6E, and cm2F.

Melon fruit-associated gene expression mediated by the promoters of the invention may be (1) ethylene regulated, (2) induced by changes in ethylene concentration in the plant, and/or (3) activated, or primarily activated, during later stages of fruit development and/or early stages of fruit ripening.

Exemplary melon fruit-associated promoters include cmACO1/TE4 (SEQ ID NO:41), MEL7 (SEQ ID NO:42), MEL2 (SEQ ID NO:43), 6E (SEQ ID NO:44) and 2F (SEQ ID NO:45).

The invention further provides plant expression vectors. Such plant expression vectors include a melon promoter of the invention operably linked to a heterologous nucleic acid coding sequence and control sequences recognized by a host cell transformed with the vector.

The invention also provides plant cells and mature plants comprising such plant expression vectors. In a related aspect, the invention provides plant cells and mature plants comprising a melon promoter of the invention.

In another aspect, the invention provides a method of expressing a heterologous nucleic acid sequence in plant cells and plants by transforming plant cells with a nucleic acid construct comprising a melon promoter of the invention operably linked to a heterologous nucleic acid coding sequence, culturing the plant cells in medium containing a selection agent to select for transformed plant cells and growing the plant cells into a transgenic fruit bearing plant.

In one aspect, the heterologous nucleic acid coding sequence is preferentially expressed in the fruit of said transgenic fruit-bearing plant. In one exemplary embodiment, the heterologous nucleic acid coding sequence is sam-k and the mature fruit of the fruit-bearing plant exhibits a decrease in ethylene production relative to a non-transgenic plant.

These and other objects and features of the invention will become more fully apparent when the following detailed description is read in conjunction with the accompanying figures and examples.

### BRIEF DESCRIPTION OF THE FIGURES

All sequences presented in the figures are shown in the conventional 5′ to 3′ orientation.

FIGS. 1A and B present the complete nucleotide sequence of the cmACO1 promoter (SEQ ID NO:40), depicted in single-stranded form where the numbering begins at the cmACO1 transcriptional start. The TATA box begins at −33 and other components of interest are indicated in the figure, e.g., adjacent vector sequences (pCR2.1).

FIG. 2A and B present the complete nucleotide sequence of the cmACO1/TE4 fusion promoter (SEQ ID NO:41), which has the tomato E4 5′UTR substituted for the native cmACO1 5′ UTR. The sequence is depicted in single-stranded form, and numbering begins at the tomato E4 transcriptional start. The TATA box begins at −47. Other components of interest are marked appropriately, e.g. restriction sites and adjacent vector sequences (pUC- 19). The cmACO1 promoter is fused to the tomato E4 5′ untranslated region at a BamlE site engineered near the transcriptional start site for each gene. An Ncol site (CCATGG) surrounds the E4 translational start site.

FIGS. 3A-C present the complete nucleotide sequence of the MEL7 promoter (SEQ ID NO:42), depicted in single-stranded form where the numbering begins at the MEL7 transcriptional start. The TATA box begins at −44 and other components of interest are indicated in the figure, e.g., restriction sites and adjacent vector sequences (pCR2.1). The NEB 1233 primer sequence is indicated by underlining the template sequence. The cmDruNcoSt primer sequence is shown as a separate opposite strand sequence. Lowercase lettering indicates an introduced nucleotide mismatch between the primer to the template.

FIGS. 4A-C present the complete nucleotide sequence of the MEL2 promoter (SEQ ID NO:43), depicted in single-stranded form where the numbering begins at the where the numbering begins at the MEL2 translational start because the transcriptional start is not known. The TATA box begins at −83 and other components of interest are marked appropriately, e.g. adjacent vector sequences (pCR2.1). The NEB 1233 primer sequence is indicated by underlining the template sequence. The MEL2_Nco_R primer sequence is shown as a separate opposite strand sequence. Lowercase lettering indicates an introduced nucleotide mismatch between the primer to the template.

FIGS. 5A-D depict the results of Northern blot hybridization of melon ripening-associated transcripts 6E and 2F. RNA was isolated from melon fruit at (1) the no-net stage; (2) the net stage; (3) prior to fruit abscission (pre-slip); (4) at one quarter slip; (5) full slip, harvest indicator; (6) over-ripe fruit; and (7) leaf tissue. Ten micrograms of RNA was separated by agarose gel electrophoresis, transferred to a nylon membrane, and probed with a labeled DNA fragment corresponding to either the 6E (5A and B) or 2F (5C and D) transcript. An autoradiograph is depicted in the upper panel of each set (5A and C) while the lower panel (5B and D) represent the relative signal intensity in each lane after scanning and signal quatitation.

FIGS. 6A and B are a single-stranded depiction of the 6E promoter (SEQ ID NO:44), where numbering begins at the 6E translational start. The TATA box begins at −100, and other components of interest are indicated in the figure, e.g., restriction sites and adjacent vector sequences (pCR2.1). The NEB 1233 primer sequence is indicated by underlining the template sequence. The 6EMELNcoP primer sequence is shown as a separate opposite strand sequence. Lowercase lettering indicates an introduced nucleotide mismatch between the primer to the template.

FIGS. 7A-C provide a single-stranded depiction of the 2F promoter (SEQ ID NO:45), operably linked to a heterologous coding sequence, e.g.GUS, as in pAG164. The numbering begins at the 2F translational start. Other components of interest are indicated in the figure, e.g., restriction sites and adjacent vector sequence (pUC-19).

FIG. 8 shows the results of an analysis for transcript abundance using gene fragments representing promoter-associated transcripts (6E, 2F, ACO1, MEL2, MEL7) along with other ripening-associated genes and controls (mCTR, SAMase, actin, and 18S rRNA) as the target DNA which was probed with a labeled cDNA probe from ripe melon fruit expressing the gene encoding SAMase (HMX8002). The relative hybridization signal strength from the melon targets is normalized to hybridization signal strength for actin in the figure.

### DETAILED DESCRIPTION OF THE INVENTION

### I. Definitions

As used herein, the term “polynucleotide” refers to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil and thymidine.

Polymeric molecules include double and single stranded ribonucleic acids (RNA) and deoxyribonucleic acids (DNA), and may include polymers having backbone modifications such methylphosphonate linkages.

A nucleic acid may be double stranded, single stranded, or contain portions of both double stranded and single stranded sequence. The depiction of a single strand also defines the sequence of the other strand and thus also includes the complement of the sequence which is depicted.

As used herein, the term “recombinant nucleic acid” refers to nucleic acid, originally formed in vitro, in general, by the manipulation of nucleic acid by endonucleases, in a form not normally found in nature.

As used herein, the terms “chimeric gene construct”, “chimeric nucleic acid construct”, “heterologous gene construct” and “heterologous nucleic acid construct” are used interchangeably and refer to recombinant nucleic acid sequences which comprise a DNA coding sequence and control sequences required for expression of the coding sequence in a plant cell.

A “heterologous” nucleic acid construct or sequence has a portion of the sequence which has been introduced into the plant cell in which it is expressed. Heterologous, with respect to a control sequence may refer to a control sequence (ie promoter or enhancer) that does not function in nature to regulate the same gene the expression of which it is currently regulating. Generally, heterologous nucleic acid are introduced into the cell or part of the genome in which they are present, and have been added to the cell, by transfection, microinjection, electroporation, or the like. The sequences may contain a control sequence/DNA coding sequence combination that is the same as, or different from a control sequence/DNA coding sequence combination found in the native plant.

As used herein, the terms “promoter” or “promoter segment” refer to a sequence of DNA that functions in a promoter disclosed herein to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

By “plant promoter” is meant a promoter or promoter region (as defined above), which in its native form, is derived from plant genomic DNA. The melon fruit-associated promoters of the invention are plant promoters.

As used herein, the term “regulatable promoter” refers to any promoter whose activity is affected by specific environmental or developmental conditions (e.g., a tomato E4 or E8 promoter).

As used herein, the term “constitutive promoter” refers to any promoter that directs RNA production in many or all tissues of a plant transformant at most times.

As used herein, the term “tissue-associated promoter” refers to any promoter which directs RNA synthesis at higher levels in particular types of cells and tissues (e.g., a fruit-associated promoter).

As used herein, “promoter strength” refers to the level of promoter-regulated expression of a heterologous gene in a plant tissue or tissues, relative to a suitable standard (e.g., a fruit-associated promoter from a particular plant, such as melon, versus a control or standard gene promoter, for example, the 35S CaMV promoter or the CsVMV promoter (Cassava Vein Mosaic Virus promoter, Verdaguer et al., 1998). Expression levels can be measured by linking the promoter to a suitable reporter gene such as GUS (>glucuronidase). Expression of the reporter gene can be easily measured by fluorometric, spectrophotometric or histochemical assays (Jefferson, et al., 1987a; Jefferson, 1987b; Jefferson, RA, 1989).

As used herein, the term “operably linked” relative to a chimeric gene or nucleic acid construct, or a heterologous gene or nucleic acid construct or vector means nucleotide components of the construct or vector are in a functional relationship with one another. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences that are linked are contiguous, and, in the case of a secretory leader, contiguous and in reading phase. However, enhancers need not be contiguous.

As used herein, the term “gene” means the segment of DNA involved in producing a polypeptide chain, which may or may not include regions preceding and following the coding region, e.g.5′ untranslated (5′UTR) or “leader” sequences and 3′UTR or “trailer” sequences, as well as intervening sequences (introns) between individual coding segments (exons).

The term “gene”, may be used interchangeably herein with the term “nucleic acid coding sequence”, and the term “structural gene” which means a DNA coding region.

As used herein, the term “sequence identity” means nucleic acid or amino acid sequence identity in two or more aligned sequences, aligned using a sequence alignment program. Sequence searches are preferably carried out using the BLASTN program when evaluating the of a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul et al., 1997.]

The term “% homology” is used interchangeably herein with the term “% identity” herein and refers to the level of nucleic acid or amino acid sequence identity between two or more aligned sequences, when aligned using a sequence alignment program. For example, as used herein, 70% homology means the same thing as 70% sequence identity determined by a defined algorithm, and accordingly a homologue of a given sequence has greater than 80% sequence identity over a length of the given sequence. Exemplary levels of sequence identity include, but are not limited to, 80%, 85%, 90%, 95% and 98% or more over a length of the given sequence.

A preferred alignment of selected sequences in order to determine “% identity” between two or more sequences, is performed using the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate stringency hybridization and wash conditions. Exemplary conditions include hybridization conducted as described in the Bio-Rad Labs ZetaProbe manual (Bio-Rad Labs, Hercules, Calif.), expressly incorporated by reference herein. For example, hybridization is conducted in 1 mM EDTA, 0.25 M Na₂HPO₄ and 7% SDS at 60( C, followed by washing in 1 mM EDTA, 40 mM NaPO₄, 5% SDS, and 1 mM EDTA, 40 mM NaPO₄, 1% SDS. Hybridization conditions are further recited in Ausubel FM et al, 1993, expressly incorporated by reference herein. An example of high stringency conditions includes hybridization at about 42° C. in 50% formamide, 5× SSC, 5× Denhardt's solution, 0.5% SDS and 100 μg/ml denatured carrier DNA followed by washing two times in 2× SSC and 0.5% SDS at room temperature and two additional times in 0.1× SSC and 0.5% SDS at 42° C.

As used herein, the terms “transformed”, “stably transformed” or “transgenic” refer to a plant cell that has a non-native (heterologous) nucleic acid sequence integrated into its genome which is maintained through two or more generations.

As used herein, the term “expression” refers to the process by which a polypeptide is produced based on the nucleic acid sequence of a gene. The process includes both transcription and translation.

As used herein, the term “modulate” refers to a change in biological activity. Modulation may relate to an increase or a decrease in biological activity, binding characteristics, or any other biological, functional, or immunological property of the molecule.

As used herein, the term “ethylene regulated”, refers to regulation which is induced by changes in ethylene concentration in the plant. For example, promoter activity which occurs or primarily occurs, during later stages of fruit development and/or early stages of fruit ripening, is said to be ethylene regulated.

As used herein, a “plant cell” refers to any cell derived from a plant, including undifferentiated tissue (e.g., callus) as well as plant seeds, pollen, progagules and embryos.

### II. Melon Promoter Isolation

Differential screening has been used to identify abundant transcripts in developing and ripening fruit. In banana, for example, a cDNA clone encoding a putative thaumatin-like protein was identified as among the most abundant transcripts in ripening fruit, and in kiwifruit a met allothionein-like transcript was identified as very abundant in ripening fruit (Clendennen et al., 1997; Ledger et al., 1995). Abundant transcripts have also been identified in the fruit of grape, cherry, and apple using differential screening methods (Tattersall et al., 1997; Fils-Lycaon et al., 1996;Lee et al., 1993).

PCR accessible CDNA libraries were made using Clontech's Marathon cDNA Amplification Kit [Clontech Laboratories, Inc., Palo Alto, Calif.], following the manufacturer's protocol. Briefly, after first and second-strand cDNA synthesis, adaptors were ligated to the polished ends of the double-stranded cDNA. This cDNA library served as a PCR-accessible cDNA library for rapid amplification of cDNA 5′ or 3′ ends.

After the libraries were constructed, modified adaptors were ligated to the digested DNA fragments, such that the adaptor defined one end of the PCR template. A pair of nested gene specific primers and a pair of nested adaptor primers were used to amplify a specific product or products.

Upstream sequences associated with either a known mRNA sequence (ACOl and MEL2), an mRNA sequence identified by RAP screening (MEL7) or by differential display of melon mRNAs (6E and 2F) were identified in a series of steps. Oligonucleotide primers were designed based on the starting sequence and used to walk upstream in a PCR-accessible melon genomic library.

A. Rap Screening

A PCR-based method to rapidly identify and clone transcripts that are abundant in a particular mRNA pool has been developed and is called random amplification of products or “RAP”-screening. RAP-screening involves random amplification of products from a serially diluted cDNA pool, followed by hybridization of amplified fragments with radiolabeled cDNA to identify abundant transcript fragments. The fragments are then easily cloned for further characterization such as sequencing and verification of expression pattern. (See Example 2.)

In carrying out RAP screening, a PCR-accessible cDNA library is constructed, diluted, and amplified with non-specific primers. Amplification products that represent high-abundance transcripts in the mRNA pool are identified by hybridization with a labeled total cDNA probe. Briefly, tissue mRNA is isolated and reverse transcribed into double-stranded cDNA. The molecules are blunt-ended and adaptors ligated to the double-stranded cDNA. The cDNA library is serially diluted 10-fold, at least ten times with the goal of reducing non-abundant transcripts to undetectable levels. Each serial dilution is used as a template in replicated PCR reactions. The primers used to amplify products are an oligonucleotide complimentary to the adaptor sequence and a cDNA synthesis primer, hence the random nature of the amplification. The replicated reactions do not give rise to identical amplification products due to the chaotic nature of the amplification reaction. The products of the PCR reactions are separated by agarose gel electrophoresis and Southern transferred, generally yielding between one and twenty distinct products from each reaction, that are visible on an ethidium bromide-stained gel. The blots are probed with radiolabeled first-strand cDNA, from the same mRNA used to construct the library. Amplification fragments which hybridize strongly to the labeled cDNA probe represent abundant transcripts in the tissue from which the RNA was extracted. Subsequent screening is required to determine the tissue or stage specificity of the identified transcripts given that the method does not include an initial differential comparison. Amplified products which strongly hybridize to the probe are cloned and their expression pattern further analyzed by Northern hybridization.

### III. Compositions and Methods of the Invention

The present invention provides regulatory/promoter sequences from a number of genes in melon that are expressed primarily or exclusively in the fruit. The melon genes are also expressed primarily in association with fruit ripening. The upstream regulatory regions described herein as cmACO1, cmACO1/TE4, MEL7, MEL2, cm6E, and cm2F promoters were demonstrated to promote reporter gene expression in ripe fruit in transient assays (Example 4).

A. cmACO1 and cmACO1/TE4 Promoters

ACC oxidase (ACO) catalyzes the conversion of ACC to ethylene in the plant biosynthetic pathway, and the enzyme is usually encoded by a multigene family. For example, three ACO genes from tomato and four, including a pseudogene, from petunia have been described (Holdworth et al. 1988; Tang et al., 1993). The ACO gene family members are generally found to be differentially expressed, usually in a tissue-specific manner or in response to wounding, senescence, or ethylene treatment. In melon the ACO gene family is represented by three members; cmACO1, cmACO2 and cmACO3 are distinguished by both nucleotide sequence and gene structure. In addition, the three genes are differentially expressed (Lasserre et al., 1996). The transcript associated with cmACO1 is the only ACO gene expressed in ripening fruit and is also induced in leaves by both wounding and ethylene treatment (Lasserre et al., 1996; Bouquin et al, 1997). A cDNA clone encoding ACOI from melon (pMELI) has been isolated and characterized (Balague et al., 1993). A melon ACO1 gene fragment has also been isolated (Lasserre et al., 1996, GenBank Accession X95551). The published 2.4 kb genomic sequence of cmACO1 includes the entire coding sequence (four exons and three introns) and approximately 739 bp of sequence upstream of the transcriptional start site (827 bp upstream of the translational start site). A 726 bp fragment of the upstream regulatory region of cmACO1 has been shown to promote expression of a reporter gene in transgenic tobacco leaves in response to wounding, ethylene treatment, and in association with natural senescence (Lasserre et al., 1997; Bouquin et al., 1997). In contrast, 5′ truncation of the cmACO1 promoter to 476 bp has been shown to abolish the wound-inducible promoter activity, but promoter activity remains inducible by ethylene (Bouquin et al., 1997).

The present invention includes the isolation and characterization of an upstream region associated with the melon ACO 1 gene that is significantly longer than previously reported sequences (Example 1). The promoter fragment was approximately 1.35 kb and corresponds to position −1256 in the established numbering system, extending to the translational start site at +101, and was designated the ACO1 promoter (SEQ ID NO:40). In one exemplary use, the 1.35 kb melon ACO1 promoter was fused to the translational start codon of an operably linked coding sequence, resulting in strong reporter gene expression in ripe melon fruit in transient assays. However, the results of stable transformations in melon, indicated low level transcription of the linked coding sequence, and also resulted in the down-regulation of native ACO1 transcription.

In order to address the issue of low level transcription, a modified promoter was constructed which is a fusion of the melon ACO1 promoter fragment from −1256 to +7 and the 17 nt tomato E4 5′UTR (GenBank accession S44898) with a 6nt linker composed of a BamfI restriction site (Example 1). The fusion promoter, designated cmACO1/TE4 (SEQ ID NO:41), is 1283 bp in length and positioned directly adjacent to the translational start site of the operably linked coding sequence. Although the mechanism is not part of the invention, post-transcriptional gene silencing is believed to be mediated by aberrant transcript fragments with homology to native genes and by substituting the heterologous tomato E4 (TE4) 5′UTR in place of the native ACO1 5′UTR in the fusion promoter, the transcript derived from the introduced expression cassette, the sequence should not exhibit homology to the native melon ACO1 gene.

B. The MEL7 and MEL2 promoters

RAP-screening was used to isolate a particularly abundant transcript fragment from ripe melon fruit. The isolated transcript (melrapF =MEL7, GenBank Accession Z70522) was shown to be relatively fruit-specific and ripening-associated by Northern blot analysis. The transcript fragment was cloned and sequenced, and using gene-specific sequence information, upstream regulatory regions were amplified from melon genomic DNA. A second, related fruit-specific gene was identified from reports in the literature (MEL2, GenBank accession Z70521), and a corresponding upstream regulatory region was obtained using sequence information from the published cDNA clone. See Example 2.

C. The Melon 6E and 2F Promoters

Differential display was used to identify transcripts specific to ripening melon fruit with the goal of isolating the associated promoters for use in controlling expression of heterologous genes specifically in the fruit and during ripening. Several differential display products were identified with the appropriate expression pattern. After the fragments of interest were cloned and further characterized, sequence specific primers were designed in order to amplify and isolate upstream genomic fragments to use as promoters. The differential display fragments were isolated, and their associated coding sequences and promoters were shown to be novel plant sequences, encoding and regulating the expression of proteins of unknown function. The fruit ripening-associated genes and promoters are designated herein as 6E and 2F (Example 3).

### IV. Generation of Transgenic Plants

A. Vectors for Transforming Plant Cells

The present invention provides heterologous nucleic acid constructs or vectors suitable for the transformation of plants and useful for the expression of heterologous genes. The invention further provides transgenic plants, transgenic plant cells and transgenic fruit, carrying such a heterologous nucleic acid construct of the invention or sequences derived form the heterologous nucleic acid construct.

The melon fruit-associated promoters of the invention find utility in heterologous nucleic acid constructs for the fruit-associated expression of genes operably linked to the promoters. The methods and results described herein are directed to gene expression under the control of the melon fruit-associated promoters of the invention, in transgenic plant cells. The melon fruit-associated cmACO1, cmACO1/TE4, MEL7, MEL2, cm6E, and cm2F promoters include a region of DNA that promotes transcription of a gene operably linked thereto, in transformed plant cells.

Using known, routine DNA manipulation techniques such as those described in Sambrook et al. (1989), heterologous nucleic acid constructs were made whereby a heterologous DNA sequence encoding a gene product of interest, is placed under the regulatory control of a melon fruit-associated promoter of the invention.

Techniques for the construction of expression vectors or heterologous nucleic acid constructs suitable for transformation into plants are generally known in the art. (See, for example, Houck et al., 1990, and Becker et al., 1992).

For expression in plants, the expression vectors of the invention contain an insertion site for a DNA coding sequence of interest. The transcription of the inserted DNA is placed under the control of a melon fruit-associated promoter of the invention.

Such expression vectors may have single or multiple transcription termination signals at the 3′ end of the DNA sequence being expressed. The expression cassette may also include, for example, (i) a DNA sequence encoding a leader peptide (e.g., to allow secretion or vacuolar targeting), (ii) translation termination signals, (iii) selectable marker genes for use in plant cells, (iv) sequences that allow for selection and propagation in a secondary host, such as an origin of replication and a selectable marker sequence.

Selectable marker genes encode a polypeptide that permits selection of transformed plant cells containing the gene by rendering the cells resistant to an amount of an antibiotic that would be toxic to non-transformed plant cells. Exemplary selectable marker genes include the neomycin phosphotransferase (nptII) resistance gene, hygromycin phosphotransferase (hpt), bromoxynil-specific nitrilase (bxn), phosphinothricin acetyltransferase enzyme (BAR) and the spectinomycin resistance gene (spt), wherein the selective agent is kanamycin, hygromycin, geneticin, the herbicide glufosinate-ammonium (“Basta”) or spectinomycin, respectively.

Typical secondary hosts include bacteria and yeast. In one embodiment, the secondary host is Escherichia coli, the origin of replication is a colE1-type, and the selectable marker is a gene encoding neomycin resistance, e.g., nptII. Origin of replication and selectable marker sequences operative in secondary hosts are well known in the art and many are commercially available (e.g., Clontech, Palo Alto, Calif.; Stratagene, La Jolla, Calif.).

The vectors of the present invention are useful for fruit tissue-associated expression of nucleic acid coding sequences in plant cells. For example, a selected peptide or polypeptide coding sequence is inserted in an expression vector of the invention operably linked to a cmACO1, cmACO1/TE4, MEL7, MEL2, cm6E, or cm2F promoter sequence. The vector is then transformed into progenitor plant cells and the plant cells are cultured under conditions to allow the expression of the protein coding sequence in the cells of a plant. Transformed plant progenitor cells may be used to produce transgenic fruit-bearing plants.

Further, the invention includes a method for producing a transgenic fruit-bearing plant, where fruit produced by the plant has a modified phenotype. In this method a heterologous nucleic acid construct is introduced (e.g., by transformation) into progenitor cells of the plant. An exemplary heterologous gene construct is composed of (i) a DNA sequence encoding a gene product effective to modify a phenotypic characteristic of the plant, e.g., to reduce ethylene biosynthesis in fruit produced by the plant, operably linked to (ii) a melon promoter of the invention, the expression of which is fruit-associated. The DNA sequence is heterologous to the promoter and the heterologous nucleic acid construct contains the appropriate regulatory elements necessary for expression in a plant cell. The method further includes transforming plant progenitor cells with a vector containing a selectable marker and a heterologous gene operably linked to a melon promoter of the invention. Transformed progenitor cells are selected by culture in the presence of a selection agent, then they are grown to produce a transgenic plant. In a preferred aspect, the transgenic plant is a fruit-bearing plant and the transgene is expressed in the fruit.

It will be understood that the vectors described herein may form part of a plant transformation kit. Components of the kit typically include, but are not limited to, reagents useful for plant cell transformation, such as vectors, Agrobacterium, reagent for transformation and culture of plant tissue, etc.

B. Methods for Transforming Plant Cells

A heterologous gene construct containing a melon fruit-associated promoter of the invention, e.g., cmACO1, cmACO1/TE4, MEL7, MEL2, cm6E or cm2F promoter may be transferred to plant cells by any of a number of plant transformation methodologies, including Agrobacterium-based methods [Ranier et al., 1990 (rice); McCormick et al., 1986 (tomato); Norelli et al., 1996 (apple)], electroporation, microinjection, or microprojectile bombardment. (See, e.g., Comai et al., 1993; Klein et al., 1988; Miki et al. 1987; Bellini et al., 1989).

In one embodiment, a heterologous nucleic acid construct is introduced into a plant by way of a T-DNA-less Ti plasmid carried by Agrobacterium tumefaciens, followed by co-cultivation of the A. tumefaciens cells with plant cells. In such cases, vectors for use in the invention contain a selectable marker gene, T-DNA border regions from Agrobacterium tumefaciens, a heterologous gene of interest, and other elements as desired. Exemplary Agrobacterium transformation vectors are commercially available from Clontech (Palo Alto, Calif.) and further described by An et al., 1985.

Other suitable vectors may be constructed using the promoters of the present invention and components of standard plant transformation vectors, which are available both commercially (Clontech, Palo Alto, Calif.) and from academic sources [Salk Institute, Plant Biology Labs; Texas A & M University; Waksman Institute, Rutgers, The State University of New Jersey, Piscataway, N.J.].

DNA may be introduced into plant cells by microprojectile bombardment using microparticles loaded with DNA which are bombarded into the cells using “gene gun” technology. (See, e.g., Robinson et al., 1997.)

Transformed plant cells obtained as a result of transformation with a heterologous nucleic acid construct comprising a melon fruit-associated promoter of the invention are cultured in medium containing the appropriate selection agent to identify and select for plant cells which express a heterologous nucleic acid sequence. After plant cells that express the heterologous nucleic acid sequence are selected, whole plants may be regenerated from transgenic plant cells. Techniques for regenerating whole plants from transformed plant cells are generally known in the art.

The invention further includes a method for producing a transgenic fruit-bearing plant. In this method a heterologous nucleic acid construct, typically carried in an expression vector allowing selection in plant cells, is introduced into progenitor cells of a plant. These progenitor cells are then grown to produce a transgenic fruit bearing plant.

Preferred plants suitable for transformation using the melon fruit-associated promoters of the invention include but are not limited to, melon, apple, tomato, pineapple, grape, raspberry, strawberry, kiwi fruit, avocado, mango, papaya, peach, pear, cherry, citrus, date palm, plantain, soybean, cotton, alfalfa, oilseed rape, flax, sugar beet, sunflower, potato, tobacco, maize, wheat, rice, nuts and lettuce.

In one exemplary embodiment, cotyledon explants of a commercial cantaloupe variety (Cucumis Melo, Muskmelon) are transformed according to known methods (Fang et al., 1990; Valles et al, 1994; Dong et al., 1991; Gonsalves et al., 1994; Yoshioka et al, 1992; Ayub et al., 1996), using a disarmed Agrobacterium strain to introduce the binary vectors into plant cells. The disarmed Agrobacterium strain is co-cultivated with melon cotyledon tissue explants, and primary transformants are selected on the basis of their capacity to regenerate and develop roots on media containing the antibiotic, kanamycin.

In other exemplary embodiments, known or modified Agrobacterium transformation methods are used to transform plant cells using the melon fruit-associated promoters of the invention. Agrobacterium transformation has been previously described for rice, tomato, apple, almond, asparagus, avocado, broccoli, carrot, cauliflower, celery, cucumber, grape, persimmon, and spinach. See, e.g., Sagi et al., 1995 (banana); Ranier et al., 1990 (rice); McCormick et al., 1986 (tomato), Van Eck et al., 1995 (tomato); Norelli et al, 1996 (apple); Miguel et al, 1999 (almond); Cabrera-et al., 1997, Delbreil B et al., 1993 (asparagus); Mogilner et al., 1993 (avocado); Hosoki et al., 1991 (broccoli); Hardegger et al., 1998 (carrot); Bhalla et al., 1998 (cauliflower); Catlin et al., 1988 (celery); Sarmento et al., 1992 and Trulson et al., 1986 (cucumber); Scorza et al., 1995 and Franks et al., 1998 (grape); Nakamura Y et al., (persimmon); and Zhang et al., 1999 (spinach).

C. Heterologous Genes for Expression using Melon Promoters of the Invention

Any structural gene of interest may be placed under the regulatory control of the melon fruit-associated promoters of the invention. The structural gene may encode a polypeptide of interest or other gene product. Of particular interest are genes associated with fruit ripening, e.g., a gene derived from an E. coli bacteriophage T3 gene that encodes a functional S-adenosylmethionine hydrolase, or SAMase protein (Hughes, J. A. et al, 1987a; Hughes et al., 1987b). SAM is a ubiquitous nucleotide used in many activities in all cells (Salvatore et al 1977; Usdin et al., 1979). SAM acts as a co-factor in a variety of reactions and as a methyl group donor in specific transmethylation reactions. Among these reactions are the biosynthesis of biotin, rare nucleotides, 5′-methylthioadenosine (MTA), polyamines and the production of the plant hormone ethylene. SAM also acts as a methyl donor during modifications of proteins, lipids, polysaccharides and nucleic acids. The SAMase protein catalyzes the conversion of S-adenosylmethionine (SAM) to methylthio-adenosine and homoserine, both of which are recycled in separate pathways. (Gold et al, 1964). In the course of ethylene biosynthesis, 1-aminocyclopropane-1-carboxylic acid (ACC), the immediate precursor to ethylene, is produced from SAM by the enzyme ACC synthase (Kende, 1993). As the pool of SAM is depleted by the action of SAMase, neither ACC nor ethylene is produced.

While the mechanism is not part of the invention, the use of a fruit-specific, developmentally regulated SAM degradation strategy as a means to reduce ethylene biosynthesis in plants has a number of distinct advantages. The fruit-specific nature of gene expression targets only the SAM found in fruit and diverted to ACC for ethylene production. This means that normal ethylene biosynthetic processes as well as other SAM-dependent reactions in the remainder of the plant proceed uninhibited. The developmental regulation of SAMase or sam-k gene expression allows for the initial climacteric burst of ethylene necessary in climacteric fruit, e.g., cantaloupe for initiation of the ripening process prior to down regulation of ethylene (Clendennen, 1999; Good, 1994). The result is a fruit of higher quality due to a lack of immaturity in the crop. The fact that sam-k gene expression follows the normal pattern of expression of ethylene during the ripening process means that the SAMase protein is essentially transient and final concentrations in the ripe fruit are minimal. An enzyme that degrades SAM may be used to allow for the selection of a broad range of modified ripening phenotypes which can be predictably determined by the level of SAMase protein expressed, resulting in an array of predictable phenotypes, which allows for production flexibility, a reduction in production and handling related losses as well as production of a higher quality crop with concomitant savings in labor and distribution costs. The lack of an immediate ripening response due to environmental effects such as increased temperature may provide a further benefit to producers, in addition to a longer-lasting, higher-quality product. Production of S-adenosylmethionine hydrolase (SAMase) in fruit alters the ethylene biosynthetic pathway and causes a modified fruit ripening phenotype in cantaloupe.

Several novel fruit-specific and ripening associated promoters have been isolated from melon. The melon promoters drive expression of heterologous genes in a fruit-specific and ripening-associated manner in transient assays in cantaloupe fruit.

In practicing the methods of the present invention, a gene derived from E. coli bacteriophage T3 encoding an enzyme capable of degrading S-adenosylmethionine (SAM), designated “SAMase” has been introduced into the Cucumis melo genome using standard Agrobacterium binary vectors. In one exemplary application, the SAMase encoding transgene is derived from a previously reported M13 clone (Hughes et al., 1987a) modified to contain a consensus eukaryotic translation initiation site by altering the nucleotide sequence surrounding the sam ATG start codon (Good et al., 1994). In practicing the invention, the SAMase gene may be placed under the control of the cmACO1, cmACO1/TE4, MEL7, MEL2, cm6E, and cm2F melon promoters.

The melon promoters described herein are suitable for the fruit-specific and ripening associated expression of the gene encoding SAMase, but are also useful for controlling expression of other heterologous genes. Exemplary genes are involved in ethylene biosynthesis (e.g., antisense ACC oxidase, antisense ACC synthase, ACC deaminase, or SAM decarboxylase) and pathogenesis-related genes such as polygalacturonase inhibiting protein (PGIP), glucanase and chitinase. Additional exemplary DNA coding sequences include, but are not limited to, sequences which encode, thaumatin, sucrose phosphate synthase and lycopene cyclase.

### V. Identification and Evaluation of Transformants

Following transformation, transgenic plant cells are assayed for expression of the transgene which is operably linked to a melon fruit-associated promoter of the invention. Transgenic plant cells may be initially selected by their ability to grow in the presence of a selective agent, such as the aminoglycoside antibiotic, or kanamycin.

Expression of a transgene may be evaluated by analysis of DNA, mRNA, and protein, associated with the expression of the transgene. In addition, where the transgene is involved in the regulation of ethylene, ethylene production by fruit from transgenic plants and/or indirect indictors of fruit ripening may be used to evaluated transgene expression using controls appropriate to the particular assay.

A. Evaluation of Promoter Activitv using Reporter Constructs

The relative activity of the melon fruit-associated cmACO1, cmACO1/TE4, MEL7, MEL2, cm6E, and cm2F promoters of the invention was evaluated in a transient assay system using a reporter gene, exemplified by GUS (glucuronidase), which is effective to evaluate the tissue-associated regulatable expression from the promoters. Expression of GUS protein is easily measured by fluorometric, spectrophotometric or histochemical assays (Jefferson, 1987a).

The recombinant nucleic acid constructs identified in Table 1 were prepared using the isolated promoter sequences described herein and techniques routinely employed in the art, then introduced into melon plant cells by particle bombardment.

TABLE 1
Melon Promoter-Containing Nucleic Acid Constructs

| | | | | sam-k | GUS |
|---|---|---|---|---|---|
| | | clone in | sam-k | expression | reporter |
| Promoter | Size | pCR2.1 | construct | construct | construct |
| cmACO1 | 1.4 kb | pAG-314 | pAG-120 | pAG-7192 | pAG-152M |
| cmACO1::TE4 | 1283 bp. | N/A | pAG-182 | pAG-4236 | pAG-158 |
| MEL2 | 2.0 kb | pAG-355 | N/A | N/A | pAG-165 |
| MEL7 | 1.9 kb | pAG-321 | pAG-181 | pAG-4238 | pAG-154 |
| 6E | 0.9 kb | PAG-353 | pAG-124 | pAG-7133 | pAG-163 |
| 2F | 2.4 kb | pAG-354 | pAG-125 | pAG-4235 | pAG-164 |

The promoter activity of various GUS constructs was then evaluated in transient assays for GUS expression. Gold particle suspensions of each construct were prepared and used to bombard sterilized melon, apple, pear and tomato fruit as detailed in Example 4.

B. Methods of Detecting Melon Promoter-Driven Gene Expression

Transgenic plants are typically assayed for their ability to synthesize mRNA, DNA and/or protein associated with expression of the coding sequence introduced into plant cells under the control of a melon fruit-associated promoter. The assays are typically conducted using various plant tissue sources, e.g., leaves, stem, or fruit.

Gene amplification and/or expression may be measured in a sample directly, for example, by conventional Southern blotting, Northern blotting to quantitate the transcription of mRNA, dot blotting (DNA analysis), or in situ hybridization, using an appropriately labeled probe, based on the sequences provided herein.

Alternatively, protein expression, may be evaluated by immunological methods, such as immunohistochemical staining of cells, tissue sections or immunoassay of tissue culture medium, e.g., by Western blot or ELISA. Such immunoassays can be used to qualitatively and quantitatively evaluate expression of the transgene. The details of such methods are known to those of skill in the art and many reagents for practicing such methods are commercially available.

A purified form of the gene product may be used to produce either monoclonal or polyclonal antibodies specific to the expressed protein for use in various immunoassays. (See, e.g., Harlow and Lane, 1988). Exemplary assays include ELISA, competitive immunoassays, radioimmunoassays, Western blot, indirect immunofluorescent assays and the like. In general, commercially available antibodies and/or kits may be used for the quantitative immunoassay of the expression level of the gene product is it is a known protein.

The following examples illustrate, but are in no way intended to limit the scope of the present invention.

### Materials and Methods

A. DNA Plasmids and Agrobacterium Binary Vector Construction

Biological reagents were typically obtained from the following vendors: 5′ to 3′ Prime, Boulder, Colo.; New England Biolabs, Beverly, Mass.; Gibco/BRL. Gaithersburg, Md.; Promega, Madison, Wisc.; Clontech, Palo Alto, Calif.; and Operon, Alameda, Calif.

Specific reagents employed in the particle bombardment include BioRad Biolistic PDS-1000/He system (BioRad Laboratories, Hercules, Calif., USA), gold particles of 1.5-3.0 μm (Aldrich, Milwaukee, Wisc., USA), a rupture disk: 1,100 PSI (BioRad Laboratories, Hercules, Calif., USA), stop screens of 0.685 mesh (Rumsey-Loomis, Freeville, N.Y.), macrocarriers: (Rumsey-Loomis, Freeville, N.Y.) and X-Gluc: 5-Bromo-4-chloro-3-indoyl β-D-glucuronide cyclohexylamine salt (Rose Scientific, Edmonton, Alberta, Canada).

Standard recombinant DNA techniques were employed in all constructions (Adams et al., 1977; Ausubel et al., 1992; Hooykaas et al., 1985; Sambrook et al., 1989), expressly incorporated by reference herein.

B. GUS Reporter Transient Assavs in Melon, Apple, Pear and Tomato

Specific equipment and reagents employed in particle bombardment included BioRad Biolistic PDS-1000/He system (BioRad Laboratories, Hercules, Calif., USA), gold particles of 1.5−3.0 μm (Aldrich, Milwaukee, Wisc., USA), a rupture disk: 1,100 PSI (BioRad Laboratories, Hercules, Calif., USA), stop screens of 0.685 mesh (Rumsey-Loomis, Freeville, N.Y.), macrocarriers: (Rumsey-Loomis, Freeville, N.Y.) and X-Gluc: 5-Bromo-4-chloro-3-indoyl-D-glucuronide cyclohexylamine salt (Rose Scientific, Edmonton, Alberta, Canada).

Solutions for use in GUS assays included: 50% Glycerol (vol/vol); 2.5M calcium chloride (CaCl₂, 13.875 grams anhydrous CaCl₂ dissolved in 50 mls sterile diH₂O); 0.1M spermidine (0.1452 grams dissolved in 10 mls sterile diH₂0); 70% EtOH (vol/vol), 3 mls sterile diH₂O in 7 mis 200 proof ethyl alcohol; X-gluc solution (200 ml prepared by adding the components in the amounts shown in Table 2, below, to 198 ml distilled H₂O, stirring for 10 minutes or until dissolved, adjusting the pH to 7.0, dissolving 100 mg X-gluc in 2 ml DMSO, adding X-gluc/DMSO solution to the pH 7.0 solution, rinsing the X-gluc vial twice using the pH 7.0 solution, and filter sterilizing the resultant solution).

TABLE 2
Solution Components

| Component | Amount | Final Conc. |
|---|---|---|
| EDTA, Disodium salt | 0.744 g | 10.0 mM |
| NaH₂PO4 · H₂O monobasic, | 1.760 g | 100.0 mM |
| monohydrate | | |
| K₄Fe(CN)₆3 H₂O | 0.042 g | 0.5 mM |
| Triton X-100 | 0.200 ml | 0.1% |

Gold particle suspensions were prepared by adding 30 of gold particles (1.5 μm to 3.0 μm) to a high quality microcentrifuge tube followed by addition of 1 ml 70% EtOH. The suspension is vortexed for 20 seconds and left to stand for 25 minutes, allowing the particles to settle to the bottom of the tube so that they do not stick to the side of the tube when centrifuging, followed by centrifuging in a microcentrifuge for 6 minutes at 13,000 rpm. The supernatant was carefully removed, discarded and 500 μl sterile diH₂O added to the tube which was vortexed for 10 seconds and left standing for 25 additional minutes, followed by centrifuging in a microcentrifuge for 6 minutes at 13,000 rpm. The supernatant was carefully removed and discarded, 500 pl sterile 50% glycerol stock added and the mixture vortexed until the particles were resuspended.

DNA solutions containing the GUS recombinant nucleic acid constructs were prepared by adding 50 μl (1 μg/μl) DNA to the microcentrifuge tube containing the gold and gently vortexing for 2-3 seconds, followed by adding 500 μl cold CaCl₂ (2.5M), gently vortexing for 2-3 seconds, adding 200 μl cold spermidine (0.1 M) and gently vortexing at low speed at 4° C., tapping the tube a couple of times every 5-10 minutes to make sure particles remained suspended, with a total vortex time of about 40 minutes. The centrifuge was pulsed to a maximum of 1,500 rpm in a microcentrifuge at 4° C. three times, the supernatant removed and discarded. 1 ml cold 70% EtOH was then added, the solution mixed and the pulse centrifuge step repeated with the supernatant removed and discarded. This pulse centrifuge step was again repeated using cold 100% EtOH, followed by adding 350 μl cold 100% EtOH and resuspending the particles by gently vortexing for 2 seconds.

Tissues were prepared for particle bombardment, as follows. Fruit slices were surface sterilized using ethanol and bleach, where fruit slices are wiped with a towel soaked in 95% ethyl alcohol, placed in a beaker to which is added an amount of a water/soap mixture sufficient to cover the samples, shaken intermittently for 10 minutes, rinsed with diH₂O until the soap is gone. 75% EtOH is added to cover the fruit slices, which are shaken gently each minute for 4 minutes, the EtOH is drained off, followed by the addition of sufficient 10% bleach/2 drops Tween 20/1000 ml to cover the samples and intermittent shaking for 10 minutes. The bleach is drained off, the samples rinsed 3 times with sterile diH₂O and once with sterile 500 ml diH₂/2 ml PPM mix (Plant Preservative Mixture, Plant Cell Technology, Washington, D.C.). The samples were cut and bombarded on the same day.

After cutting, melon fruit was plated onto medium containing: MS salts, MS vitamins, 3% Sucrose, 1 mg/L BAP, 50 mg/L myo-Inositol, 2 ml/l PPM, 25 mg/l Cefotaxime (aa) and 0.8% Agar at a pH of 5.8. After cutting, apple and pear fruit was plated onto medium containing: MS salts with B5 vitamins, glycine (2 mg/L), sucrose (30 g/L), Casein hydrolysate (100 mg/L), BAP (6-benzyl aminopurine, 0.5 mg/L), 2,4-D (1.5 mg/L), PPM (plant preservation mixture, 5 ml/L), ascorbic acid (100 mg/L), citric acid (100 mg/L), cefotaxime (200 mg/L), pH 5.8 and solidified with phytagel (2.5 g/L), for one or two days prior to microprojection.

Leaves from in vitro rooted plants were segmented, placed on medium containing: N6 salts, B5 vitamins, glycine, sucrose, casein hydrolysate, cefotaxime (100 mg/L), TDZ (Thidiazuron 1 or 3 mg/L) and IAA or IBA (2 mg/L), solidified with phytagel (2.5 g/L), and bombarded on the same day.

After cutting tomato fruit was plated onto medium containing: MS salts, MN vitamins, 3% Sucrose, 0.5 mg/l Zeatin and 0.7% Agar at a pH of 5.8.

The fruit tissue was bombarded using GUS reporter constructs, a flight distance of 6 cm and a PS1 of 1,100. Flight distance is defined as the distance between the DNA coated microcarrier and stopping screen to the target cells. PSI refers to the helium pressure in the gas acceleration tube used for particle bombardment. After the fruit tissue was bombarded, it was sealed with parafilm and left in the dark at 24° C. for 22 hours, then explants were carefully transferred to clean, sterile petri plates and X-gluc solution added to completely cover the fruit. Plates were stored in an incubator at 37° C. for 18 hours, then the X-gluc solution was drained off and 95% EtOH added to cover the fruit. Observations were made using a microscope and counting the number of GUS foci on each slice of fruit.

### EXAMPLE 1

Melon cmACO1 and cmACO1/TE4 Promoters

A. Isolation and Characterization of cmACO1 and cmACO1/TE4 Promoters

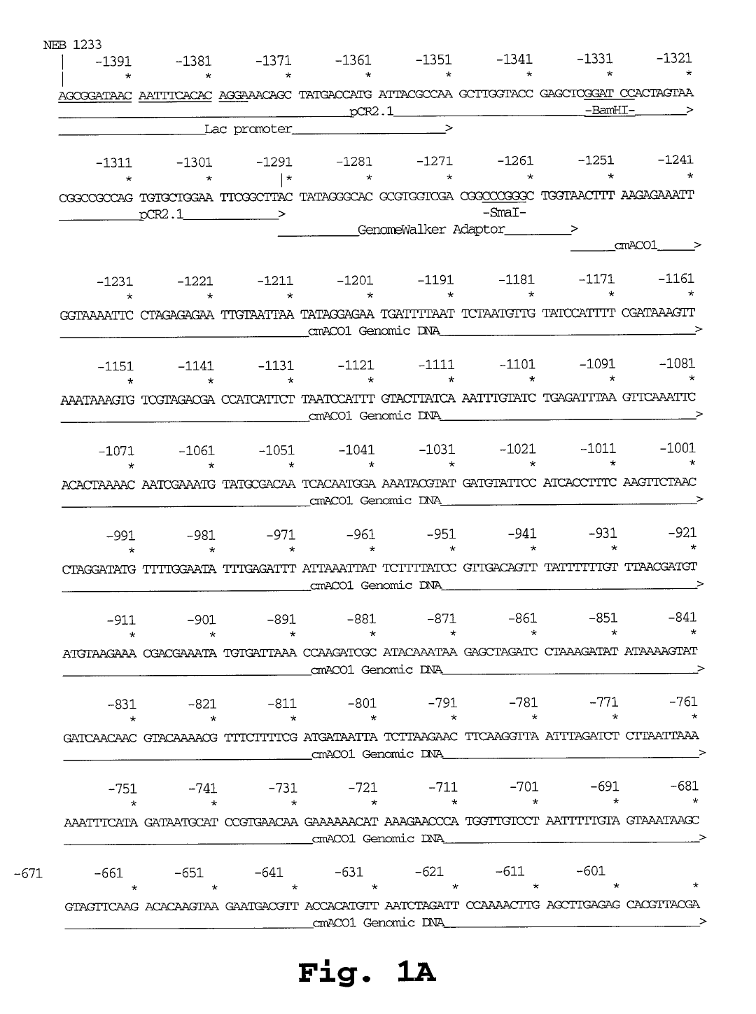

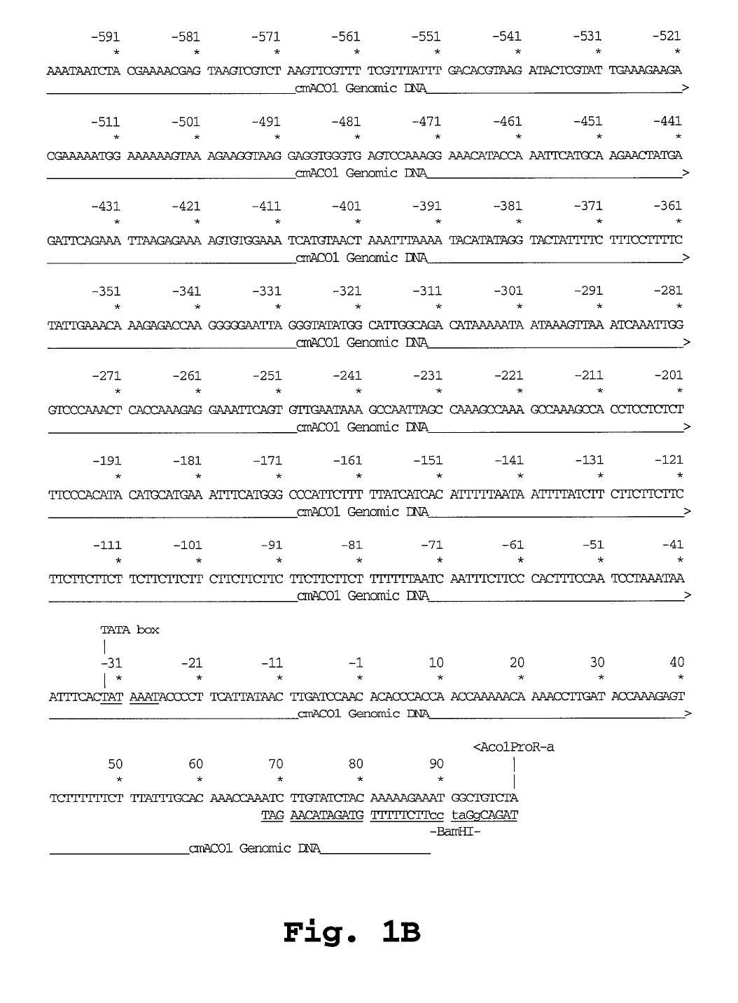

The present invention includes the isolation and characterization of an upstream region associated with the melon ACO1 gene that is significantly longer than previously reported sequences. The isolated promoter fragment is approximately 1.35 kb and corresponds to position −1256 in the established numbering system and extends to the translational start site at +101, designated the ACO1 promoter. FIGS. 1A and B present the complete nucleotide sequence of the cmACO1 promoter (SEQ ID NO:40).

Clontech Universal GenomeWalker libraries were constructed and screened according to the supplier's protocol (Clontech Laboratories, Inc., Palo Alto, Calif.). Each PCR-accessible genomic library is created by digestion with a restriction enzyme that leaves blunt ends. Following the protocol five restriction enzymes are used in five separate reactions. The digested genomic DNA is then purified. After purification the digested genomic DNA is ligated overnight to GenomeWalker adaptor-1 (SEQ ID NO: 1). Following ligation, the ligase is heat inactivated, the ligation reaction diluted and two rounds of PCR are performed to amplify unknown sequences adjacent a known sequence. Using GenomeWalker primer AP1 (SEQ ID NO:3), the distal of the two GenomeWalker adaptor primers, and a primer that can bind to a known sequence, (a first gene-specific primer), PCR is performed using each of the different GenomeWalker libraries as templates. Each of the primary reactions are then diluted and used as a template for secondary PCR reactions. The primers used in the secondary reaction are GenomeWalker AP2 (SEQ ID NO:4), and a second, internal, gene specific primer.

A cDNA clone of ACO1 from Cucumis melo was first identified by its homology to the gene encoding ACO1 in tomato (Balague et al., 1993). Based on the nucleotide sequence of the cmACO1 cDNA, sequence-specific oligonucleotide primers PFACO1 #I (SEQ ID NO:5) and PFACO1 #2 (SEQ ID NO:6) were designed to walk upstream in melon PCR-accessible genomic libraries (Universal Genome Walker Kit, Clontech Laboratories, Inc., Palo Alto, Calif.), prepared as described above.

After two rounds of amplification of the melon libraries, a 1.4 kb product was amplified from the EcoRV library (library 2). Products were present in other libraries, however, they were all smaller than 1.4 kb. The 1.4 kb fragment was cloned into pCR2.1 and was designated pAG-314. The cmACO1 GenomeWalker product overlapped with the cmACO1 cDNA by an exact match of 43 nucleotides.

A Basic BLASTN search (http://www.ncbi.nlm.nih.gov/BLAST/) of non-redundant nucleic acid sequence databases through NCBI (http://www.ncbi.nlm.nih.gov/index.html) indicated that the 1.4 kb cmACO1 promoter sequence corresponds to a portion of the sequence found in GenBank at Accession Number X95551.

A 2.4 kb genomic melon ACO1 gene sequence has been reported, which includes the entire coding sequence (four exons and three introns) and approximately 739 bp of sequence upstream of the transcriptional start site or 827 bp upstream of the translational start site (Lasserre et al., 1996, GenBank accession X95551). A 726 bp fragment of the upstream regulatory region of cmACO1 was shown to promote expression of a reporter gene in transgenic tobacco leaves in response to wounding, ethylene treatment, and in association with natural senescence (Lasserre et al., 1997; Bouquin et al., 1997). In contrast, a 5′ truncation of the cmACO1 promoter to 476 bp was shown to abolish the wound-inducible promoter activity, but promoter activity remains inducible by ethylene (Bouquin et al, 1997), suggesting the presence of a wound-responsive element between −726 and −476, and an independent ethylene responsive element between −476 and +1.

The present invention is directed to the isolation of an upstream region associated with the melon ACO1 gene that is significantly longer than that previously reported. The melon ACO1 promoter fragment described herein is approximately 1.35 kb in length and corresponds to position −1256 in the established numbering system extending to the translational start site at +101.

In one exemplary use, the 1.35 kb melon ACOl promoter was fused to the translational start codon of an operably linked coding sequence, resulting in strong reporter gene expression (e.g., GUS) in ripe melon fruit in transient assays. However, the results of stable transformations in melon, indicate that the cmACO1 promoter results in low level transcription of the linked coding sequence, and also results in the down-regulation of native ACO1 transcription.

Accordingly, the fusion promoter, designated cmACO1/TE4 (SEQ ID NO:41), is 1283 bp in length and positioned directly adjacent to the translational start site of the operably linked coding sequence. Although the mechanism is not part of the invention, post-transcriptional gene silencing is believed to be mediated by aberrant transcript fragments with homology to native genes and by substituting the heterologous tomato E4 (TE4) 5′UTR in place of the native ACO1 5′UTR in the fusion promoter, the transcript derived from the introduced expression cassette should not exhibit homology to the native melon ACO1 gene.

In a second exemplary use, a modified promoter (cmACO1/TE4) was constructed which is a fusion of the melon ACO1 promoter fragment from −1256 to +7 and the 17nt tomato E4 5′UTR (GenBank accession S44898) with a 6nt linker composed of a BamHI restriction site (Example 1; SEQ ID NO:41). In constructing the fusion promoter, the melon ACO1 promoter fragment from −1256 to +7 was fused to the 17nt tomato E4 5′UTR (GenBank Accession S44898) with a 6nt linker composed of a BamHI restriction site. The resulting fusion promoter is 1283 bp and is directly adjacent to the translational start site for an operably linked coding sequence. More specifically, the 5′UTR associated with the ACO1 promoter was replaced with the tomato E4 gene 5′UTR, an alternative 5′UTR with no homology to melon ACO1. The tomato E4 5′UTR was first fused to the sam-k coding sequence. The tomato E4 5′UTR is only 17 bp long and so it was assembled by annealing two oligonucleotides, E4UTR5 ′UP (SEQ ID NO:9) and E4UTR5′LO (SEQ ID NO: 10). The cohesive end of a Bamffl site was engineered into the 5′ end of the 5′UTR and an NcoI cohesive end was engineered at the 3′ end of the 5′ UTR, to facilitate cloning this fragment.

B. Subcloning the ACO1 and ACO1/TE4 Hybrid Promoters as Translational Fusions with sam-k.

The cmACO1 promoter was subcloned into an expression construct with GUS and as a translational fusion with the sam-k gene encoding S-adenosylmethionine hydrolase. The cmACO1 promoter was amplified from pAG-314 using NEB 1233 (SEQ ID NO:7) and ACO1 ProR-a (SEQ ID NO:8). In FIG. 1A, the NEB 1233 primer sequence is indicated by underlining and the ACO1ProR-a primer sequence is shown as a separate opposite strand sequence. Lowercase lettering indicates an introduced nucleotide mismatch between the primer to the template. A BamHI site was introduced near the 3′ end, as indicated under the sequence of the ACO1 ProR-a primer, in order to facilitate subdloning and to eliminate an ATG codon in the 5′ untranslated region of the cmACO1 promoter that might function as a false, out-of-frame start codon.

The PCR amplified fragment was digested with an appropriate restriction enzyme (BamHI) and cloned into compatible sites in a sam-k-containing construct, with the cmACO1 promoter operably linked with the sam-k start codon. The resulting construct was designated pAG-120.The cmACO1 promoter was also fused to a BamHI site 10 nt upstream of the ATG start codon of the GUS reporter gene, resulting in a construct designated pAG152M.

A binary plant expression construct was needed in order to make transgenic plants containing the ACO1 promoter operably linked to sam-k. To this end the cmACO1 promoter-sam-k fusion was digested from pAG-120 to produce cohesive ends (HindIII and KpnI), and the fragment was cloned into compatible sites in a binary plant expression construct. This new plasmid consisted of a selectable marker expression cassette (the RE4pro::nptII::G7terminator) and the cmACO1 promoter-sam-k fusion followed by the nos terminator between the left and right T-DNA borders of Agrobacterium tumefaciens. The resulting construct was designated pAG-7192.Melon plants transformed with pAG-7192 showed evidence of downregulation of the endogenous melon ACO1 gene.

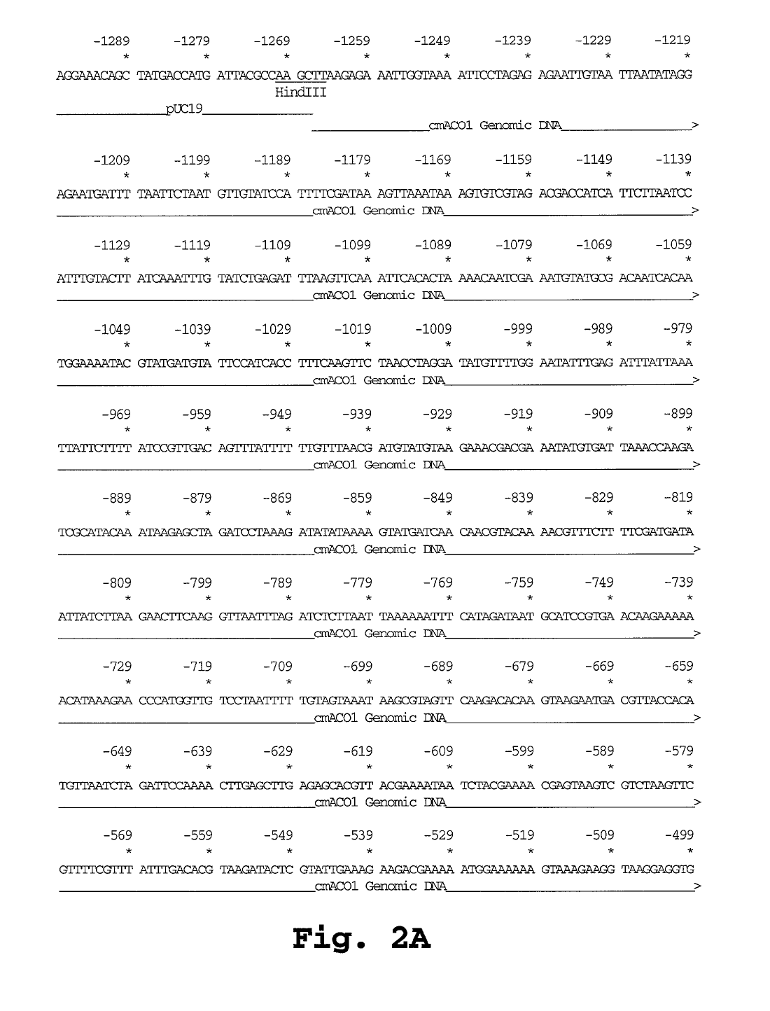

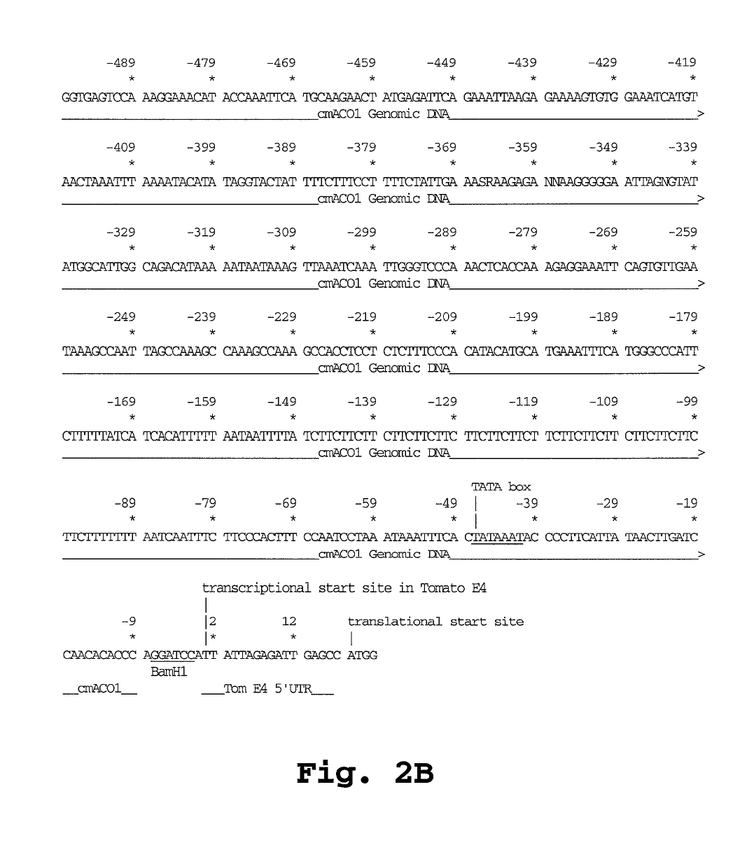

In order to introduce the cmACO 1 promoter into the TomE4::sam-k fiusion, the 1.3 kb cmACO1 promoter (SEQ ID NO:40) was amplified from pAG-314 using cmDruP5′H3 (SEQ ID NO: 11) and cmDruP3Bam (SEQ ID NO: 12). HindIII and BamHI sites were engineered into the 5′ and 3′ primers respectively. The amplified product did not include the ACO1 5′UTR. After amplification and digestion the cmACO1 promoter fragment was cloned into compatible sites in the TomE4:: sam-k fusion construct. (See FIG. 2B) The resulting construct was designated pAG-182. Finally the binary plant expression construct was made. The fragment containing the cmACO1 promoter-TomE4 5′UTR-sam-k cassette was digested from pAG-182 using HindIII and Sael and cloned into a binary plant expression plasmid. This plasmid, designated pAG-4236, consists of a selectable marker expression cassette (the RE4pro::nptII::G7 terminator) and cmACO1 promoter-TomE4 5′UTR-sam-k followed by the nos terminator between the left and right T-DNA borders of Agrobacterium tumefaciens.

C. Subcloning the cmACO1 Promoter with the GUS Reporter Gene

Another series of plasmids were made which contained the GUS reporter gene rather than sam-k. The 1.3 kb cmACO1 promoter was amplified from pAG-3 14 using NEB 1233 (SEQ ID NO:7) and ACO1ProR-a (SEQ ID NO:8). A BamHI site was engineered into the 3′ primer. The PCR amplified fragment was digested with appropriate restriction enzymes (SmaI and BamHI). (FIG. 1) The promoter was then cloned into compatible sites (PstI made blunt by reacting with Vent DNA polymerase and BamHI) in a reporter gene construct, comprised of the promoter operably linked with GUS and containing the nos terminator. The resulting construct was designated pAG-152M.

A reporter gene construct was also constructed which contained the cmACO1/TomE4 5′UTR sequence (described above). The assembled tomato E4 5′UTR fragment was cloned into compatible sites in a reporter gene construct. The 1.3 kb cmACO promoter was amplified from pAG-3 14 using cmDruP5 ′H3 (SEQ ID NO: 11) and cmDruP3Bam (SEQ ID NO: 12). The PCR amplified product was digested to produce the appropriate cohesive ends (HindIII and BamHI) and cloned into compatible sites in the tomato E4 5′UTR::GUS::Nos terminator containing plasmid. The resulting construct was designated pAG-158.

### EXAMPLE 2

### MEL7 and MEL2 Promoters

A. Isolation and Characterization of MEL7 and MEL2 Promoters

cDNA libraries were generated using RNA isolated from ripe cantaloupe fruit by the following method: Four to six grams of frozen tissue was ground to a fine powder using a mortar and pestle in liquid nitrogen. The powder was combined with 12.5 ml extraction buffer (2% CTAB, 2% PVP (K30), 100 mM Tris-HCI, pH 8. 0,25 mM EDTA, 2.0 M NaCl, 0.5 g/L spermidine, 2% -mercaptoethanol) pre-warmed to 65° C. After thorough mixing, an equal volume of chloroform:isoamyl alcohol (24:1) was added to the tissue/buffer slurry. The mixture was centrifuged and the upper aqueous phase transferred to a clean tube. One quarter volume of 10 M LiCl was added to the recovered solution and allowed to incubate at 4° C. overnight. Subsequently the sample was centrifuged, the supernatant decanted, and the pellet resuspended in SSTE (1 M NaCl, 0.5% SDS, 10 mM Tris-Cl, pH 8.0, 1 mM EDTA, pH 8.0). This RNA was extracted once more with chloroform:isoamyl alcohol (24:1) and precipitated with 2.5 volumes ethanol. The purified RNA was pelleted by centrifugation, washed with 70% ethanol, then dried and resuspended in a small volume of nuclease-free water.

Poly(A)+RNA was isolated from 600 μg total RNA using the Straight A's mRNA Isolation System Kit (Novagen, Inc., Madison, Wisc.). The library itself was made using Clontech's Marathon cDNA Amplification Kit [Clontech Laboratories, Inc., Palo Alto, Calif.] following the manufacturer's protocol. Briefly, after first and second-strand cDNA synthesis, adaptors were ligated to the polished ends of the double-stranded cDNA. This cDNA library served as a PCR-accessible library for random amplification of products (RAP) and screening by hybridization.

B. RAP Reactions and Hybridization

The melon PCR-accessible cDNA libraries were serially diluted 10-fold in TE, from 10⁻¹ to 10⁻¹⁰ of the original library concentration. The diluted libraries were used as a template for replicate PCR amplification reactions. Each 5041 reaction mix was composed of: 38.5 μl water; 5 μl 10× KlenTaq reaction buffer (Clontech, Palo Alto, Calif.); 0.5 μl of each primer (100 μM); 0.5μl dNTP mix (25mM each); 5 μg diluted library; 1 μl KlenTaq DNA polymerase (Clontech). The primers used were MAR AP1 (SEQ ID NO: 13) and CSP (SEQ ID NO: 14). The reactions were cycled in a Robocycler Gradient 96 Temperature Cycler with Hot Top Assembly (Stratagene, La Jolla, Calif.) using the following cycling parameters: 5 cycles [94° C., 30s; 72° C, 3 min], 5 cycles [94° C., 30s; 70° C., 3min], 35 cycles [94° C., 30s; 68° C., 3min], 1 cycle [72° C., 10 min], followed by a 6° C. hold. A 10 μl aliquot of each amplification reaction was separated by agarose gel electrophoresis, photographed, then transferred to nylon membrane (Nytran Plus, Schleicher and Schuell) by capillary blotting using 0.4M sodium hydroxide as the transfer fluid. The labeled first-strand cDNA probe used in RAP screening was synthesized from 0.5 μg poly(A)+mRNA in the presence of 1.5 μM α-[³²P] dCTP (3000 mCi/mmol) using an oligo(dT)₁₅ primer (Promega) and 15U MMLV reverse transcriptase (Promega) for 1 h at 42° C.

The labeled first-strand cDNA was separated from unincorporated radioisotope by G-50 column purification according to the manufacturer's instructions (ProbeQuant G-50 Micro Column, Pharmacia, N.J.). Blots were prehybridized for 30 min in 1 mM EDTA, 0.25 M phosphate buffer (pH 7.2), 7% (w/v) SDS, and hybridized overnight at 60° C. in the same solution containing the denatured probe. Hybridized filters were washed twice for 30 min each at 60° C. in Wash Solution One (1 mM EDTA, 40 mM phosphate buffer, pH 7.2, 5% (w/v) SDS) and three times for 30 min each at 65° C. in Wash Solution Two (1 mM EDTA, 40 mM phosphate buffer pH 7.2, 1% (w/v) SDS). The air-dried filters were subjected to autoradiography to visualize hybridizing fragments.

Four different transcript fragments (melrapC, E, F, and J) were detected that showed a very strong signal intensity after hybridization with labeled cDNA from ripe melon fruit, indicating that each fragment represented an abundant transcript in ripe melon fruit. The tissue distribution of the abundant melon transcripts was determined by Northern blot analysis. The transcript fragments were radiolabeled and hybridized to RNA from root, stem, and leaf tissue, flowers, unripe green fruit, and ripe orange fruit. Three of the transcripts (melrapC, F, and J) were expressed primarily in the fruit, but one fragment (melrapF) was expressed most abundantly in ripe fruit only. The melrapF fragment was cloned and sequenced, and melrapF exhibited nucleotide sequence homology to a previously characterized raspberry gene encoding a major latex-like protein (GenBank Accession AJ224145) and to a previously isolated melon cDNA clone called MEL7 (Aggelis et al., 1997; GenBank Accession Z70522). In the published report (Aggelis et al., 1997), the MEL7 cDNA clone was isolated along with three other cDNAs that increased in abundance during melon fruit ripening. One of the published cDNAs, MEL1, has been identified as encoding ACC oxidase, while MEL5 encodes phytoene desaturase. MEL2 and MEL7 have been described as encoding proteins of unknown function (Aggelis et al., 1997).

C. Walking Upstream from the melrapF/MEL7 cDNA Sequence

The melrapF transcript fragment identified by RAP screening was found to have homology to MEL7, a cDNA clone of a ripening-related mRNA (Aggelis et al., 1997) and the protein encoded by MEL7 showed some homology to the major latex protein from opium poppy. The nucleotide sequence of the melrapF/MEL7 cDNA was used to design a sequence-specific oligonucleotide primer pM7-5R-1′ (SEQ ID NO: 15) which was used to walk upstream in melon PCR-accessible genomic libraries (Universal Genome Walker Kit, Clontech Laboratories, Inc. ). The libraries were constructed and screened according to the supplier's protocol, except that in addition to the five restriction endonucleases included in the kit, five more blunt-cutters were also used to digest genomic DNA prior to adaptor ligation: Hpal, MscI, PshAI, Smal and SnaBI. After one round of amplification of the melon libraries a 1.9 kb product was amplified from the HpaI library (library 6). This product was so abundant that it was cloned immediately into pCR2.1 rather than performing the secondary reaction as is typical with GenomeWalker experiments. The plasmid containing the 1.9 kb GenomeWalker fragment was designated pAG-321. The 1.9 kb GenomeWalker fragment was sequenced and found to match the first 264 bases of the MEL7 cDNA sequence.

D. Subcloning the MEL7 Promoter into Expression Cassettes

The MEL7 promoter fragment was subcloned as a translational fuision with the reporter gene encoding GUS. For ease of cloning it was engineered with a 3′ end NcoI site and amplified directly from the GenomeWalker clone pAG-321 using NEB 1233 (SEQ ID NO:20) and cmDruNcoSt (SEQ ID NO:16). The PCR amplified MEL7 promoter fragment (1.56 kb) was digested with HindIII and NcoI to produce the appropriate cohesive ends and cloned into compatible sites in a reporter gene construct. The resulting plasmid, comprised of the promoter operably linked to GUS and followed by the nos terminator, was designated pAG- 154.

The MEL7 1.56 kb promoter fragment (SEQ ID NO:42) was also subdloned as a translational fusion with sam-k. For ease of cloning it was engineered with a 3′ end NcoI site and amplified directly from the GenomeWalker clone pAG-321 using NEB 1233 and cmDruNcoSt.

The PCR amplified MEL7 promoter-containing fragment was digested with BamHI and NcoI to produce the appropriate cohesive ends, and cloned into compatible sites in a sam-k -containing construct. The resulting plasmid, comprised of the promoter operably linked to sam-k, was designated pAG-181.

A binary plant expression construct was constructed in order to make transgenic plants containing the MEL7 promoter operably linked to sam-k. In order to prepare such a binary plant expression vector, the MEL7 promoter-sam-k fusion was digested from pAG-181 to produce cohesive ends and the fragment was cloned into compatible sites in a binary plant expression construct. The plasmid included a selectable marker expression cassette (the RE4pro::nptII::G7terminator) and the MEL7 promoter-sam-k fusion followed by the nos terminator between the left and right T-DNA borders of the Agrobacterium tumefaciens. The resulting construct was designated pAG-4238.

E. Walking Upstream from the MEL2 cDNA Sequence

The MEL2 cDNA clone of a ripening-related mRNA (Aggelis et al., 1997; GenBank Accession Z70521) encodes a putative protein which is unidentified. Based on the nucleotide sequence of the MEL2 cDNA, sequence-specific oligonucleotide primers pMel2-outer (SEQ ID NO: 17) and pMel2-inner (SEQ ID NO: 18) were designed to walk upstream in the melon PCR-accessible genomic libraries. (Universal Genome Walker Kit, Clontech Laboratories, Inc.). The libraries were constructed and screened according to the supplier's protocol, except that in addition to the five restriction endonucleases included in the kit, five more blunt-cutters were also used to digest genomic DNA prior to adaptor ligation: HpaI, MscI, PshAI, Smal and SnaBI. After two rounds of amplification of the melon libraries a 2.2 kb product was amplified from the MscI library (library 7). Products were present in other libraries but they were all smaller than 2.2 kb. This 2.2 kb fragment was cloned into pCR2.1 and was designated pAG-355.

F. Subcloning the MEL2 Promoter with the GUS Reporter Gene

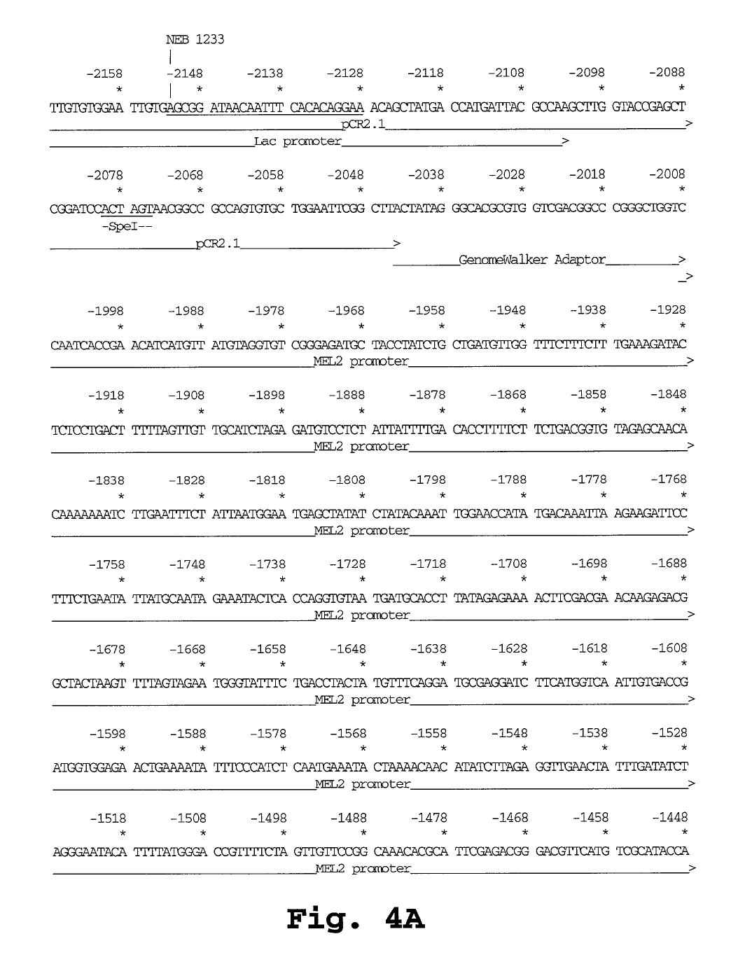

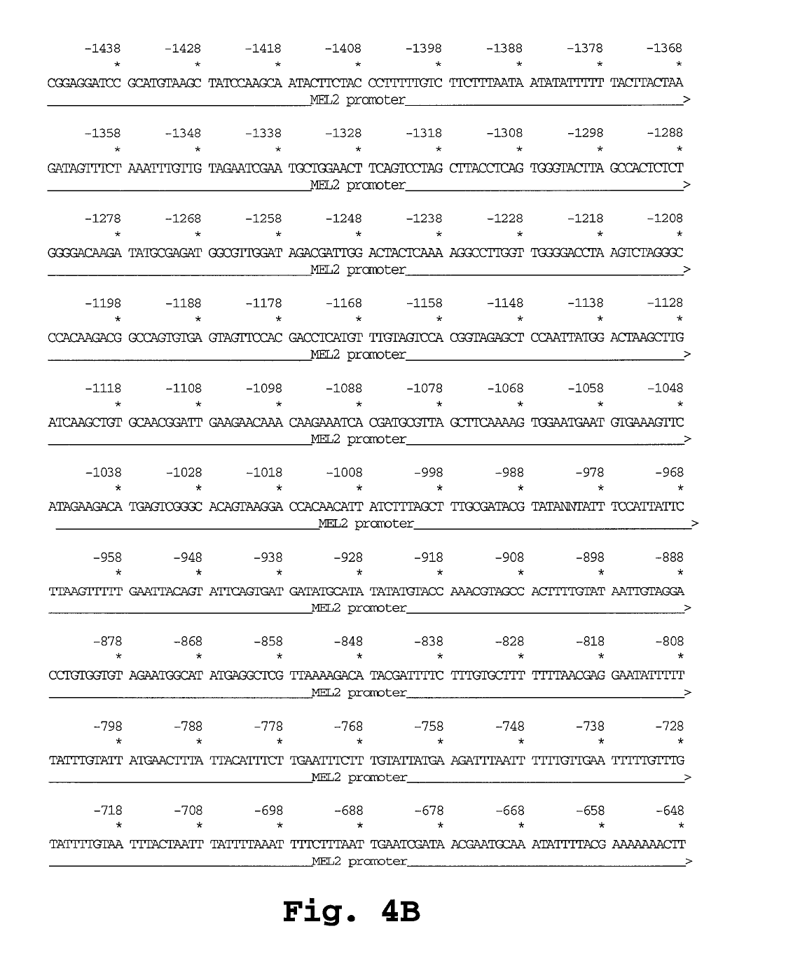

The MEL2 promoter fragment was subcloned as a translational fusion with GUS. For ease of cloning it was engineered with a 3′ end NcoI site and amplified directly from the GenomeWalker clone pAG-355 using NEB 1233 (SEQ ID NO:20) and MEL2 NcoR (SEQ ID NO:19). The PCR-amplified MEL2 promoter (2.0 kb, SEQ ID NO:43) was digested with SpeI and NcoI to produce the appropriate cohesive ends and cloned into compatible sites in a reporter gene construct (FIG. 4). The resulting plasmid, comprised of the promoter in a translational fusion with GUS and followed by the nos terminator, was designated pAG-165.

### EXAMPLE 3

### The Melon 6E and 2F Promoters

A. Isolation of Melon 6E and 2F Promoters

Differential display (U.S. Pat. No. 5,262,311; Liang et al., 1998) was used to identify transcripts specific to ripening melon fruit with the goal of isolating the associated promoters for use in controlling expression of heterologous genes specifically in the fruit and during ripening. Several differential display products were identified with the appropriate expression pattern. After the fragments of interest were cloned and further characterized, sequence specific primers were designed in order to amplify and isolate upstream genomic fragments to use as promoters. The differential display fragments were isolated and their associated coding sequences and promoters were shown to be novel plant sequences. The fruit ripening-associated genes and promoters are designated herein as 6E and 2F.

RNA was extracted from melon tissue of the San Marcos variety, including: an immature fruit (roughly 85 mm, estimated to be 20 days post pollination), and a ripe fruit (roughly 135 mm, estimated to be 45 days post pollination). The melon fruit was collected in the field and rapidly frozen in liquid nitrogen, followed by RNA extraction based on the RNA extraction method published by Chang et al., 1993, modified for melon as follows. Four to six grams of frozen tissue was ground to a fine powder in liquid nitrogen using a mortar and pestle. The powder was combined with 12.5 ml extraction buffer pre-warmed to 65° C. (2% CTAB, 2% PVP (K30), 100 mM Tris-HCl, pH 8.0, 25 mM EDTA, 2.0 M NaCl, 0.5 g/L spermidine, autoclaved for 15 minutes at 121° C., with 2% β-mercaptoethanol added just prior to use.) After thorough mixing, an equal volume of chloroform/isoamyl alcohol (24:1) was added to the tissue/buffer slurry. The mixture was centrifuged, the upper aqueous phase transferred to a clean tube and one quarter volume of 10 M LiCl added to the recovered solution and allowed to incubate at 4° C. overnight. Subsequently, the sample was centrifuged, the supernatant decanted, and the pellet resuspended in SSTE (1 M NaCl, 0.5% SDS, 10 mM Tris-Cl, pH 8.0, 1 mM EDTA, pH 8.0). The sample was then mixed with chloroform/isoamyl alcohol (24:1), centrifuged and the aqueous phase transferred to a separate clean tube and precipitated using 2.5 volumes ethanol. The precipitated RNA was pelleted by centrifugation, the pellet washed, dried and resuspended in nuclease-free water.

B. Differential Display Reactions and Analysis

The discovery of fruit-specific transcripts was made through the use differential display PCR using the GenHunter RNAimage™ kit (GenHunter Corp. Nashville, Tenn.) according to the manufacturer's instructions. Briefly, using single-base anchored oligo (dT) primers containing a Hindull restriction site (H-T11-N, where N=G, A, or C) first-strand cDNA was synthesized from 200 μg DNase-treated total RNA, using either ripe or immature melon RNA as the template. For each of the first-strand cDNA's synthesized 16 PCR reactions were performed. 2.5% of each first-strand cDNA was used as a template. The anchor primer used to make the first-strand cDNA was used as one primer in the PCR reaction along with one of the eight random primers. Each PCR reaction was carried out in duplicate to control for random errors. Included in each reaction mix was a radiolabeled nucleotide α-[33P] dATP, DuPont/NEN) to allow autoradiographic detection and the resulting products were analyzed by autoradiography. Products present in mature fruit but absent in immature fruit were identified after denaturing polyacrylamide gel electrophoresis using a large-format gel for separation, followed by autoradiography. After desired amplification products were identified they were recovered directly from the dried polyacrylamide gel by re-amplification using the original primer set. Two of the fragments identified by this method, designated E and F, were further characterized and analyzed. Fragment E was a 184 bp fragment amplified with the H-T11-A anchor primer and the H-APS random primer. Fragment F was a 217 bp fragment amplified with the H-T11 -A anchor primer and the H-AP7 random primer.

C. Analysis and Cloning of Differential Display Fragments E and F

After the differential display amplification products were recovered from the acrylamide gel and reamplified with the original primer set, the amplification products were transferred to dot blots to confirm tissue distribution of the associated transcript. Following a standard Dot Blot protocol (Nucleic Acid Dot-Blots on S&S Transfer Media, Schleicher and Schuell Laboratory Manual, 6th edition, pp. 24-25) the differential display fragments were transferred to a nylon membrane in triplicate. After the transfer was complete, the membrane was cut into three replicates. Each of the three replicates was hybridized with a different probe: radiolabeled cDNA from either immature melon fruit, mature melon fruit or melon leaf. Radiolabeled cDNA was made by reverse transcribing 30-40 μg of each mRNA from the appropriate tissue. Using standard hybridization techniques routinely employed in the art the radiolabeled cDNA was used to probe the dot blots. The mature melon probe hybridized to both the E and F fragments, while the immature melon fruit and melon leaf probes did not.

The 6E and 2F differential display products were re-amplified from the acrylamide gel and re cloned into pCR2.1 (Invitrogen, Carlsbad, Calif.) in separate reactions. In order to confirm that the differential display acrylamide gel bands contained a single DNA fragment, several independent clones from both the E and F transformations were tested to confirm their mature fruit specific expression pattern. Using a standard Northern blot protocol (Northern Max Protocol from Ambion, Austin, Tex.) 20 μg each of RNA extracted from ripe melon fruit, immature melon fruit and melon leaves was separated by agarose gel electrophoresis and transferred to a nylon membrane. The blots were hybridized separately with several of the E and F clones. The clones designated 6E and 2F detected bands in the ripe melon RNA lane of the blots but did not detect bands in the leaf and immature melon RNA lanes. The labeled 6E fragment hybridized to a transcript of approximately 1500 nucleotides, while the labeled 2F 3 fragment hybridized to a transcript of about 500 nucleotides. The pCR2.1 clones containing the 6E and 2F differential display fragments were given the designations pAG-351 and pAG-352, respectively.

Sequence analysis was performed using Sequenase® and visualized by autoradiography after separation by high-resolution electrophoresis.

D. Walking Upstream from the Cloned Differential Display Products 6E and 2F

A PCR-accessible cDNA library (Clontech Marathon cDNA Amplification Kit, Clontech Laboratories, Inc., Palo Alto, Calif.) was constructed and screened according to the supplier's protocol. Briefly, total RNA was extracted from melon fruit as previously described. Poly(A)+RNA was recovered from total RNA using Novagen's Straight A isolation system (Novagen, Inc., Madison, Wisc. After first and second-strand cDNA was synthesized from the isolated polyA+RNA, Clontech's Marathon adaptor (SEQ ID NO: 1) was ligated to the polished ends of the double-stranded cDNA. This cDNA library served as a set of templates for rapid amplification of cDNA 5′ or 3′ ends (5′ or 3′RACE, respectively).

The oligonucleotide primer, 6E-PF1 (SEQ ID NO:32), along with the Clontech Marathon Adaptor Primer, API (SEQ ID NO: 13), was used to walk upstream from the 6E differential display fragment in a ripe melon fruit Marathon cDNA Library via a 5′RACE reaction. A fragment of approximately 1.1 kb was obtained that, when sequenced, included the remainder of the 6E coding sequence and 50 bp of sequence upstream of the deduced 6E start codon. The 1.1 kb 6E 5′RACE product overlapped with the 6E differential display fragment by 61 nt, and the nucleotide sequence was an exact match.

The sequence-specific primer, 2F-PF1 (SEQ ID NO:36), along with the Clontech Marathon Adaptor Primer, AP1 (SEQ ID NO:3), was used to walk upstream in a ripe melon fruit Marathon cDNA Library via a 5′RACE reaction. A 344 bp fragment was obtained that included the remainder of the 2F coding sequence and 17 bp of sequence upstream of the deduced putative start codon. The 2F 5′RACE product overlapped with the 2F differential display fragment by approximately 120 bp, and the nucleotide sequence was an exact match.

Sequence analysis of the 5′RACE fragments was performed using Sequenase® and visualized by autoradiography after separation by high-resolution electrophoresis.

Clontech Universal GenomeWalker libraries were constructed and screened according to the supplier's protocol (Universal Genome Walker Kit, Clontech Laboratories, Inc., Palo Alto, Calif.). Based on the nucleotide sequence of the 6E differential display clone, gene-specific oligonucleotide primers 6E-PF1 (SEQ ID NO:32) and 6E-PF2 (SEQ ID NO:33) were designed to walk upstream in melon PCR-accessible genomic libraries. After two rounds of amplification of the melon libraries using 6E-PFI and AP1 in the primary reaction, and AP2 (SEQ ID NO:4) and 6E-PF2 (SEQ ID NO:33) in the secondary reaction, a 1 . 8 kb product was amplified from the Dral library (library 1). This 1.8 kb fragment was designated as the 6E promoter (SEQ ID NO:44) and cloned into pCR2.1 to generate clone pAG-353. The 6E GenomeWalker product overlapped with the 6E differential display fragment by an exact match of 34 nucleotides. The 1.8 kb 6E Genome Walker fragment overlapped with the 6E 5′RACE product by approximately 1050 bp, and included 0.9 kb of sequence upstream of the putative translational start site.

Based on the nucleotide sequence of the 2F differential display clone, gene-specific oligonucleotide primers 2F-PF1 (SEQ ID NO:36) and 2F-PF2 (SEQ ID NO:37) were designed to walk upstream in melon PCR-accessible genomic libraries. After two rounds of amplification of the melon libraries using 2F-PF1 and AP1 in the primary reaction, and 2F-PF2 and AP2 in the secondary reaction, a 3.6 kb product was amplified from the PvuIl library (library 3). Products were present in other libraries but they were all smaller than 3.6 kb. This 3.6 kb fragment was designated as the 2F promoter and was cloned into pCR2.1 to generate clone pAG-354. The 3.6 kb 2F GenomeWalker product overlapped with the 2F differential display fragment by an exact match of 59 nucleotides, and included a 2.4 kb region upstream of the putative translational start site.

Sequence analysis was performed by one of two methods: Sequenase® and visualization by autoradiography after separation by high-resolution electrophoresis, or by using cycle sequencing with fluorescent dyes and detection using the Perkin-Elmer ABI 310.

A Basic BLASTN search (http://www.ncbi.nlm.nih.gov/BLAST/) of non-redundant nucleic acid sequence databases (consisting of all non-redundant GenBank+EMBL+DDBJ+ascPDB sequences, but no Expressed Sequencing Tags (ESTs), STS, Genome Survey Sequence, or High Throughput Genomic Sequences) through NCBI (http://www.ncbi.nlm.nih. gov/index.html) revealed no significant database matches to the sequence of either the 6E or 2F promoters.

E. Northern Analysis of 6E and 2F GenomeWalker Products

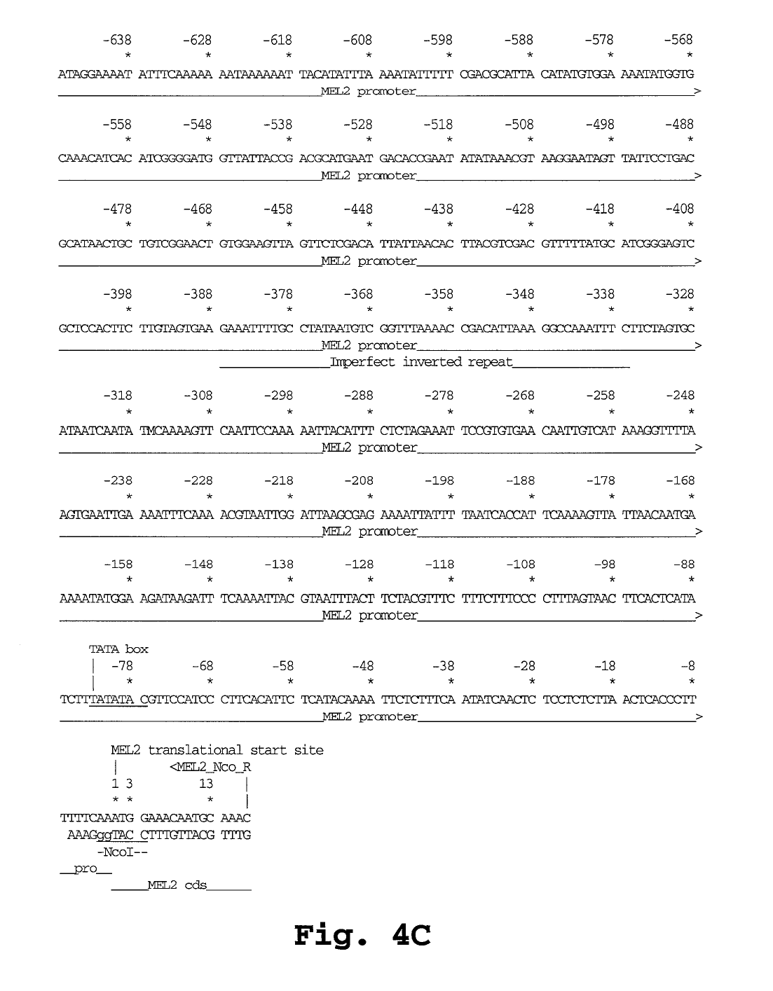

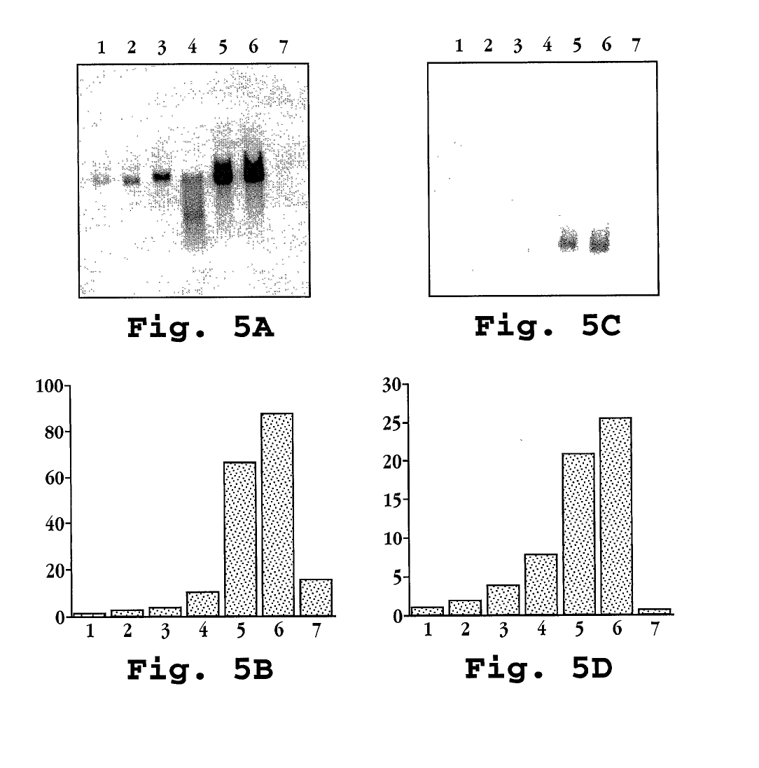

The expression patterns of 6E and 2F GenomeWalker products were confirmed by Northern blot hybridization using routine procedures. In summary, RNA was isolated from melon fruit at six different stages of development; overripe fruit, full slip, ¼ slip, pre ¼ slip, net stage, no net stage in addition to RNA isolated from melon leaves. Melon fruit development stages are defined as follows: “overripe stage” refers to a fruit past the full slip stage, where the fruit has separated from the plant, “full slip stage” refers to a mature fruit whose abscission zone between the peduncle (fruit stem) and the fruit is fully developed; “¼ slip stage” refers to an immature fruit whose abscission zone between the peduncle (fruit stem) and the fruit is one quarter developed; “pre ¼ slip stage” refers to an immature fruit between the net stage and the ¼ slip stage, where no abscission zone has developed; “net stage” refers to an immature fruit which has just developed a netted appearance (reticulum) on the exterior of the fruit and “no net stage” refers to an immature fruit which has not yet developed a netted appearance (reticulum) on the exterior of the fruit. Ten micrograms of RNA was separated by agarose gel electrophoresis and transferred to a nylon membrane. The blots were probed with digested, gel purified and labeled 6E (1200 nucleotide) and 2F (500 nucleotide) GenomeWalker fragments from pAG-353 and pAG-354. The Northern blot results indicated that the 6E and 2F transcripts are present at all stages of melon fruit development and ripening, but only at extremely low levels no net and net (FIG. 5A and 5B, respectively). Based on hybridization signal strength, the amount of transcript increased in parallel with fruit development (FIG. 5A-B).

F. Subcloning the 6E and 2F Promoters with GUS/sam-k

The 6E 0.9 kb genomic fragment upstream of the putative translational start site was subcloned as a translational fusion with both GUS and sam-k. For ease of cloning, the 3′oligonucleotide primer used to amplify the 6E promoter fragment was engineered with a 3′ NcoI site. The 6E 0.9 kb promoter fragment was amplified from pAG-353 using primers NEB 1233 (SEQ ID NO:35) and 6EMELNcoP (SEQ ID NO:34) then digested with BamHI and NcoI to produce the appropriate cohesive ends. This fragment was then cloned into compatible sites in an expression cassette with either the GUS reporter gene or the sam-k coding sequence. The expression construct, comprised of the promoter in a translational fusion with GUS and containing the nos terminator, was named pAG-163.A construct comprised of the promoter in a translational fusion with SAMase and containing the nos terminator, was named pAG-124. Finally, the 6E 0.9 kb promoter fragment operably linked to sam-k, digested from pAG-124 with SacI and Spel to produce the appropriate cohesive ends, was cloned into compatible sites in a binary plant expression construct. The resulting construct was named pAG-7133.In addition, pAG-7133 contains a selectable marker cassette consisting of a constitutive promoter, RE4, operably linked to the selection marker nptII and terminated with the G7 terminator.

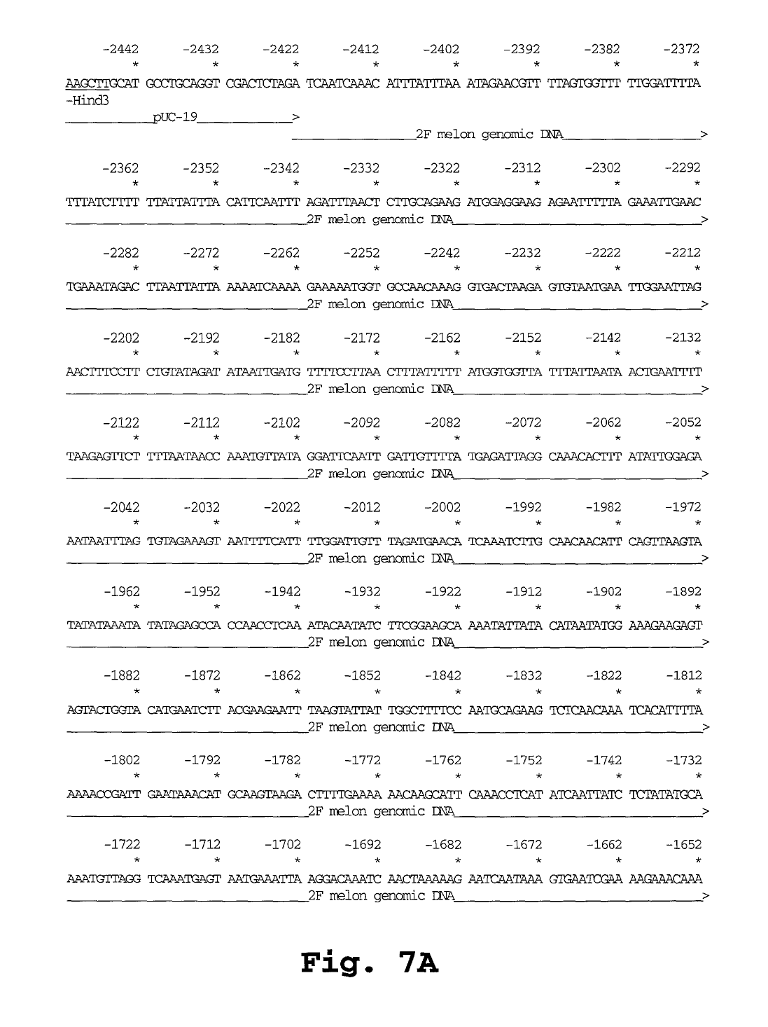

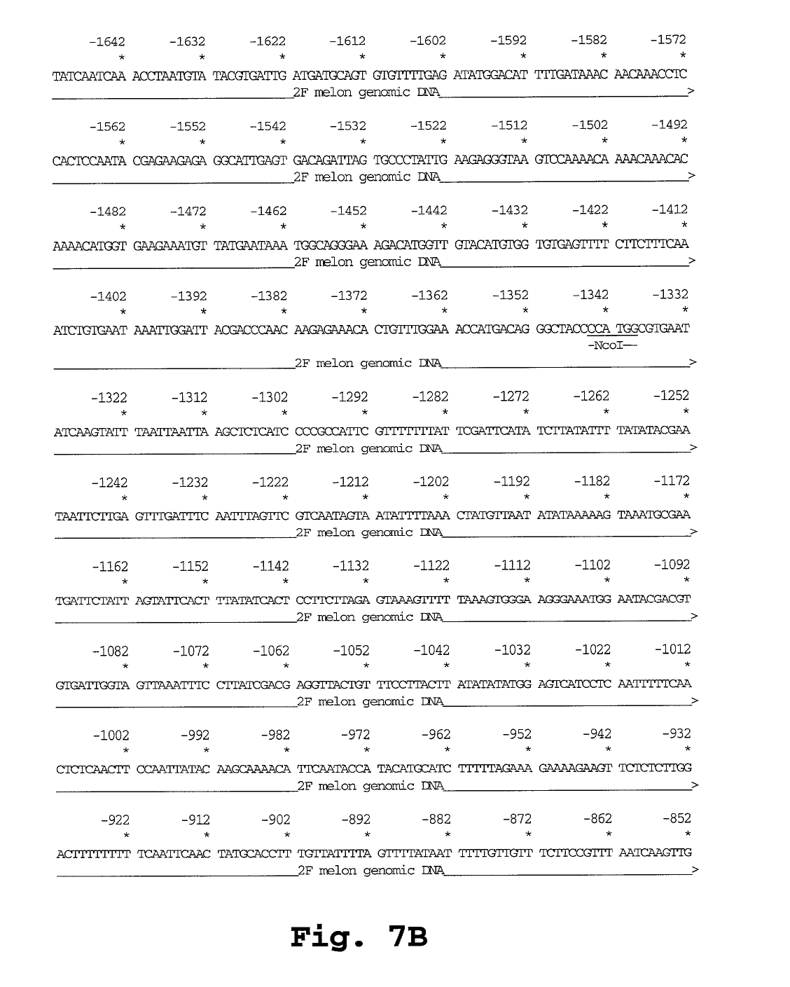

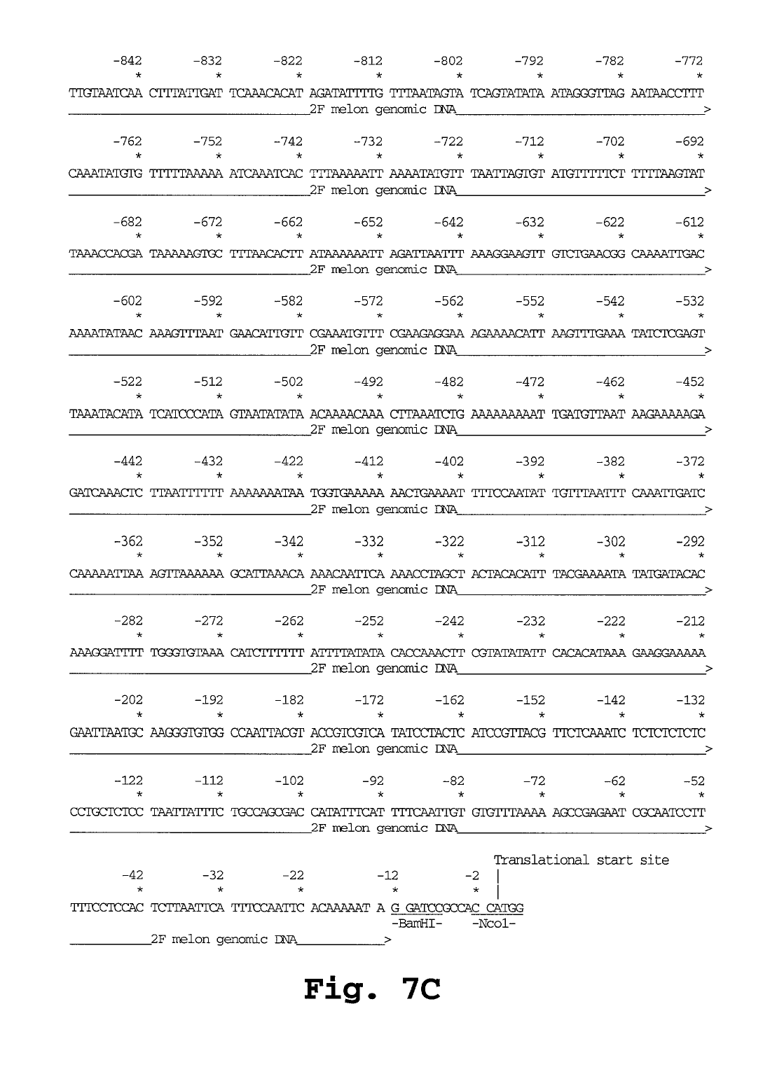

The 2F 2.4 kb promoter fragment was subcloned as a translational fusion with sam-k. For ease of cloning it was engineered with a 3′ end BamHI site and amplified directly from the Genome Walker clone pAG-354 using NEB 1224 (SEQ ID NO:39) and 2FBamStart (SEQ ID NO:38). The PCR amplified fragment was digested with EcoRV and BamHI and cloned into compatible sites in a SAM-containing construct. The resulting construct was named pAG-125. The 2F 2.4 kb promoter fragment (FIGS. 7A-C) operably linked with GUS was made by assembling the 2F melon promoter in two steps: first the 1.1 kb HindIII to NcoI distal portion of the promoter was added upstream of GUS. Next the NcoI to Ncol 1.3 kb proximal portion of the promoter was inserted between the distal portion of the promoter and GUS creating a translational fusion with GUS. The transcriptional terminator from nos is fused to GUS and the resulting construct named pAG-164. Finally, the 2F 2.4 kb promoter fragment operably linked with SAM, digested from pAG-125 to produce the appropriate cohesive ends, was cloned into compatible sites in a binary plant expression construct. The resulting construct was named pAG-4235. In addition pAG-4235 contains a selectable marker cassette consisting of a constitutive promoter, RE4, operably linked to nptII and terminated with the G7 terminator.

### EXAMPLE 4

### Analysis of Promoter Activity

A. Promoter Activity Assays in Melon Fruit

Relative promoter activity was determined for the melon promoters described in Examples 1-3: cmACO1, cmACO1 ::TE4, MEL2, MEL7, 6E and 2F. Melon promoter::GUS recombinant nucleic acid constructs were individually introduced into ripe melon fruit tissue by particle bombardment. The actin promoter which is a strong constitutive promoter was used as a positive control and a standard for relative promoter activity. The relative activity of the melon fruit-associated promoters was determined by a transient assay system using the GUS reporter gene. The transient assay is based on particle bombardment of plant tissue sections with a suspension of DNA and gold particles, and uses standard techniques (Herrera-Estrella et al., 1994). Target tissues (ripe melon fruit slices) were bombarded at 6cm flight distance with gold particles (1.5-3.0 μm) and assayed for histochemical GUS expression 24h after bombardment. Melon fruit slices were cultured under sterile conditions for 1 or 2 days prior to bombardment. The results of the GUS activity assay as a measure of promoter activity are presented in Table 3.All of the melon fruit-associated promoters result in GUS reporter gene expression at a level approximately equal to the strong constitutive actin promoter. The cmACO1 promoter and the cmACO1::TE4 fusion promoter both exhibited the same level of activity. These data suggest that the melon fruit-associated promoters described herein exhibit significant promoter activity in ripe melon fruit, the intended target tissue.

TABLE 3

| | Transient GUS Assay in Ripe Melon Fruit | | |
|---|---|---|---|
| | Total #fruit | Mean # slices | Mean # foci |
| Promoter | slices assayed | with foci (%) | per slice (SD) |
| cmACO1 | 48 | 40 (83) | 6.9 (7.2) |
| cmACO1::T | 48 | 38 (79) | 6.6 (6.9) |
| E4 | | | |
| MEL2 | 48 | 40 (83) | 6.5 (5.1) |
| MEL7 | 48 | 36 (75) | 5.0 (3.6) |
| 6E | 48 | 42 (87) | 5.9 (5.9) |
| 2F | 48 | 30 (63) | 3.4 (2.4) |
| actin | 48 | 45 (94) | 7.3 (6.1) |

B. Relative Transcript Abundance of Candidate Genes in Ripe Melon Fruit

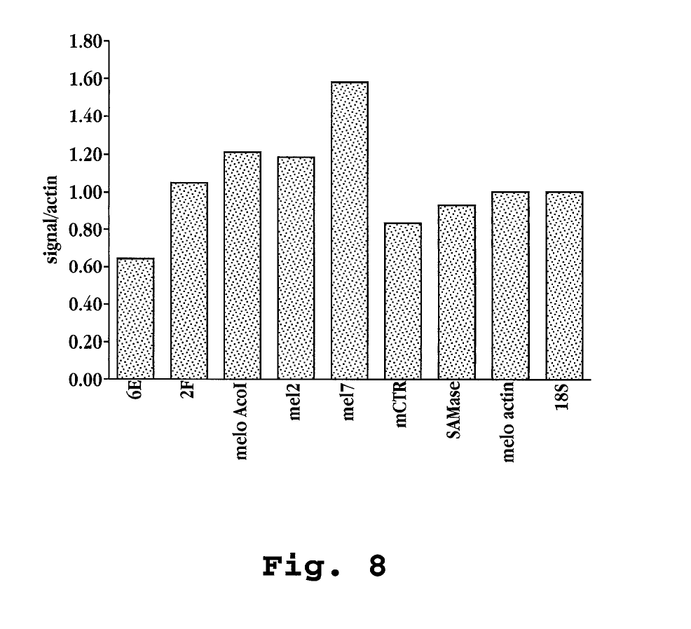

Relative transcript abundance was used to estimate the relative activity of native promoters. This was accomplished by isolating total RNA from leaf, stem, root and immature, pre-slip, slip, and post-slip fruit tissue for several melon lines. Gene fragments representing the promoter-associated transcripts 6E, 2F, ACO1, MEL2 and MEL7, along with other ripening-associated genes and controls (mCTR, SAMase, actin, and 18S rRNA) were amplified and used as targets on DNA blots. The DNA targets were probed with a labeled cDNA probe derived from ripe transgenic fruit expressing the gene encoding SAMase. FIG. 8 illustrates the relative hybridization signal strength from the melon targets nornalized to the hybridization signal for actin. These preliminary results confirm previous studies that show MEL7 as a relatively abundant transcript in ripe fruit tissue, as compared to 6E, 2F, ACO 1 and MEL2.Both ACO1 and MEL2 also exhibited promoter activity clearly above the level of the actin control.

Plant expression constructs in which the melon promoters are operably linked to the coding sequence for SAMase and reporter transgenes have been made. Preliminary evidence supports the activity of the MEL7 promoter.

C. Promoter Activity in Other Fruit

The relative activity of the cmACO 1 and MEL7 promoters was also tested in heterologous fruit tissues, including apple (Table 4), pear (Table 5) and tomato (Table 6). The method and assay are the same as described above. In Table 4, the activity of the melon fruit-associated promoters cmACO1, cmACO1/TE4, and MEL7 are compared to the strong constitutive promoter, CsVMV in mature apple fruit. In Table 5, the melon fruit associated promoter MEL7 is compared with CsVMV in mature pear fruit, and in Table 6, the melon fruit associated promoter MEL7 is compared with CsVMV in mature tomato fruit. In all cases, the relative promoter activity of the melon fruit-associated promoters in mature fruit, as measured by reporter gene expression, is comparable to the activity of the strong constitutive promoter used as a standard (CsVMV, Verdaguer et al., 1996). These data suggest that the fruit-associated promoters isolated from melon can also control heterologous gene expression in other fruit tissues.

TABLE 4

| | Transient GUS Assay in Apple Fruit | | |
|---|---|---|---|
| | Total #fruit | Mean # with | Mean # foci/ |
| Promoter | slices | GUS foci (%) | slice SD |
| cmACO1 | 18 | 3 (17) | 1 (0) |
| cmACO1/TE | 18 | 2 (11) | 1.5 (0.7) |
| 4 | | | |
| MEL7 | 48 | 6 (12) | 0.6 (0.5) |
| CsVMV | 48 | 8 (17) | 2.0 (1.7) |

TABLE 5

| | Transient GUS Assay in Pear Fruit | | |
|---|---|---|---|
| | Total #fruit | Mean # with | Mean # foci/ |
| Promoter | slices | GUS foci (%) | slice (SD) |
| MEL7 | 36 | 11 (31) | 2.4 (3.2) |
| GsVMV | 36 | 19 (53) | 3.4 (3.8) |

TABLE 6

| | Transient GUS Assay in Tomato Fruit | | |
|---|---|---|---|
| | Total #fruit | Mean # with | Mean # foci/ |
| Promoter | slices | GUS foci (%) | slice SD |
| MEL7 | 12 | 10 (83) | 13.4 (10.5) |
| CsVMV | 12 | 12 (100) | 17.0 (17.9) |

The relative activity of the melon fruit-associated promoter cmACO1/TE4 has also been tested in developing apple and pear fruit and in apple and pear leaves. Slices of immature apple fruit (2.5 cm diameter, 5-6 seeds), developing fruit (4.5 cm diameter, 3-6 seeds) mature fruit (5.5 cm diameter, 3-6 seeds) and apple leaves were assayed for GUS expression in a transient assay as directed by the melon fruit-associated promoter cmACO1/TE4 (Table 7). The activity of the cmACO1/TE4 promoter (as measured by percent of fruit slices with foci and mean number of GUS foci) was shown to increase during fruit development, and was greatest in mature apple fruit.

TABLE 7

| | Transient GUS Assay in Developing | | |
|---|---|---|---|
| | Apple Fruit and in Leaves | | |
| | Total #fruit | Mean # with | Mean # foci/ |
| Promoter | slices or leaves | GUS foci (%) | slice (SD) |
| cmACO1/TE4 | 30 | 6 (20) | 2.5 (2.4) |
| (immature fruit) | | | |
| cmACO1/TE4 | 32 | 3 (9.4) | 1 (0) |
| (developing fruit) | | | |
| cmACO1/TE4 | 32 | 10 (31) | 2 (1.5) |
| (mature fruit) | | | |
| cmACO1/TE4 | 24 | 5 (21) | 2.2 (1.6) |
| (leaves) | | | |

Immature pear fruit (2.5 cm diameter, 9-10 seeds), developing pear fruit (3.5 cm diameter, 9-10 seeds), mature pear fruit (5 cm diameter, 9-10 seeds) and leaves were also assayed for transient GUS activity as directed by the cmACO1/TE4 promoter (Table 8).

TABLE 8

| | Transient GUS Assay in Developing | | |
|---|---|---|---|
| | Pear Fruit and in Leaves | | |
| | Total #fruit | Mean # with | Mean # foci/ |
| Promoter | slices or leaves | GUS foci (%) | slice (SD) |
| cmACO1/TE4 | 30 | 5 (16.7) | 1.4 (0.5) |
| (immature fruit) | | | |
| cmACO1/TE4 | 32 | 13 (40.6) | 3.1 (3.3) |
| (developing fruit | | | |
| cmACO1/TE4 | 32 | 16 (50) | 3.8 (4.0) |
| (mature fruit) | | | |
| cmACO1/TE4 | 27 | 9 (33) | 2.3 (2.6) |
| (leaves) | | | |

Consistent with the results obtained for apple, the relative activity of the cmACO1 promoter was shown to be greater in more mature fruit, at which time the activity in fruit was greater than in the leaves. These data support the activity of a the melon promoter in controlling gene expression in mature fruit of other species, and also indicate that the promoter activity is fruit-associated.

### EXAMPLE 5

### Field Tests of Transpenic Melon Expressing SAMase Under Control Of The MEL7 Promoter

Stable transgenic plants comprising the MEL 7 promoter controlling expression of the sam-k gene which encodes SAMase were generated by introducing a heterologous nucleic acid construct which has the cmDru (MEL 7) promoter operably linked to sam-k. The vector, designated pAG423 8, includes the sam-k coding sequence under the control of the cmDru (MEL 7) promoter, a nos termination element located downstream of the sam-k coding sequence adjacent the right border of the plasmid; and the nptII selectable marker coding sequence under the control of a raspberry RE4 promoter with an Agrobacterium gene 7 termination element located downstream of the nptII coding sequence, adjacent the left border of the plasmid (cmDru::sam-k::RE4:nptII).

pAG423 was introduced into cantaloupe using cotyledon explants of a commercial cantaloupe variety (Cucumis Melo, Muskmelon) according to known methods for Agrobacterium-based transformation. A disarmed Agrobacterium strain was co-cultivated with melon cotyledon tissue explants, and primary transformants selected on the basis of their capacity to regenerate and develop roots on media containing the antibiotic, kanamycin. Following selection, transgenic plant cells were grown to transgenic plants using standard plant regeneration techniques routinely employed by those of skill in the art.

Field trial plantings of stable transgenic cantaloupe plants comprising the MEL 7 promoter controlling the expression of SAMase were carried out in Davis, California. During the Summer of 2000, a detailed analysis of the field grown transgenic cantaloupe plants was performed by evaluating the ethylene production rates in transgenic lines and their respective controls. Seed from melon transformed with the pAG4238 construct were sown in the greenhouse at Davis, Calif. Tissue samples were collected and analyzed for the presence of vector backbone, SAMase and zygosity, using techniques routinely employed by those of skill in the art. The segregation ratio was also checked to confirm that the gene was segregating in a single, dominant fashion.

After testing, plants were transplanted to the field based on nucleic acid analysis and results for seed increase and evaluation. All lines were generation T, and segregating. Only plants which were confirmed as expressing sam-k by PCR were transplanted to the field.

Ethylene data were collected from full slip fruit using formation of the abscission zone as a harvest indicator. Fruit was harvested from August 18 until September 8, 2000.Ten fruits were sampled from each line/event for ethylene testing. Ethylene testing was carried out as follows:

Plots were evaluated daily for maturity of the fruit. Fruit was considered mature (“full slip”) when it fully detached from the vine at the peduncle, forming a complete abscission zone. Mature fruit were removed from the plot, labeled and taken to the lab for ethylene evaluation. Within an hour of harvest the stem end of the fruit was capped with a rubber cone/septum and sealed with silicon. The gas in the cap was allowed to equilibrate for a minimum of 3 hours before sampling. After 3 hours, a gas tight syringe was inserted in the septum, pumped (gas drawn up in to the syringe, plunger depressed forcing the gas back into the cap, thus, mixing the gas in the cap) and a 1 milliliter sample was drawn. The sample was injected into a gas chromatograph and the results were recorded as ppm ethylene gas. The results shown in Table 9 indicate that significant differences exist between control and transgenic fruit in ethylene production.

TABLE 9
Ethylene Evolution by Fruit from Transgenic and Control Plants

| Fruit | 1 | 2 | 3 | 4 | 5 | 6 | 7 | 8 | 9 | 10 | MAX |
|---|---|---|---|---|---|---|---|---|---|---|---|
| control | 47.98 | 29.92 | 49.40 | 41.15 | 17.84 | 14.68 | 38.31 | 78.93 | 13.10 | 44.03 | 78.93 |
| Event #52 | 22.17 | 22.63 | 12.53 | 18.48 | 8.64 | 10.15 | 16.22 | 16.21 | 4.93 | 10.02 | 22.63 |
| control | 194.68 | 20.67 | 143.84 | 77.20 | 7.23 | 61.18 | 40.90 | 17.54 | 75.18 | 28.46 | 194.68 |
| Event #134 | 11.59 | 7.87 | 16.04 | 4.99 | 14.10 | 25.30 | 10.05 | 4.07 | 5.90 | 13.40 | 25.30 |

The ethylene production by non-transgenic control fruit ranged from 78. 93 to 194. 68 ppm. The average ethylene evolution from control non-transgenic fruits at later dates (towards the rear of the trial) was greater (194. 68 ppm) than towards the start of the trial (78. 93 ppm). HTransgenic events/lines identified as #52 and #134 exhibited decreases in ethylene evolution as compared to controls. In addition, the average ethylene evolution from both Event #52 and #134 was similar even though Event #134 was located in the “high” ethylene or rear portion of the trial.

The examples provided herein demonstrate that transgenic plants may be developed with modified ethylene biosynthesis, associated delayed fruit ripening which is associated with extended postharvest life in climacteric fruit. This may be accomplished using the melon promoters of the invention operably linked to an appropriate transgene, exemplified herein by SAMase. The results presented herein demonstrate that the melon promoters of the invention can regulate transgene expression in a fruit-specific and ripening-associated manner.

Transgenic fruit exhibiting such a modified ethylene ripening phenotype finds utility in reduction of post-harvest losses resulting from produce that is overripe and senescent.

TABLE 10

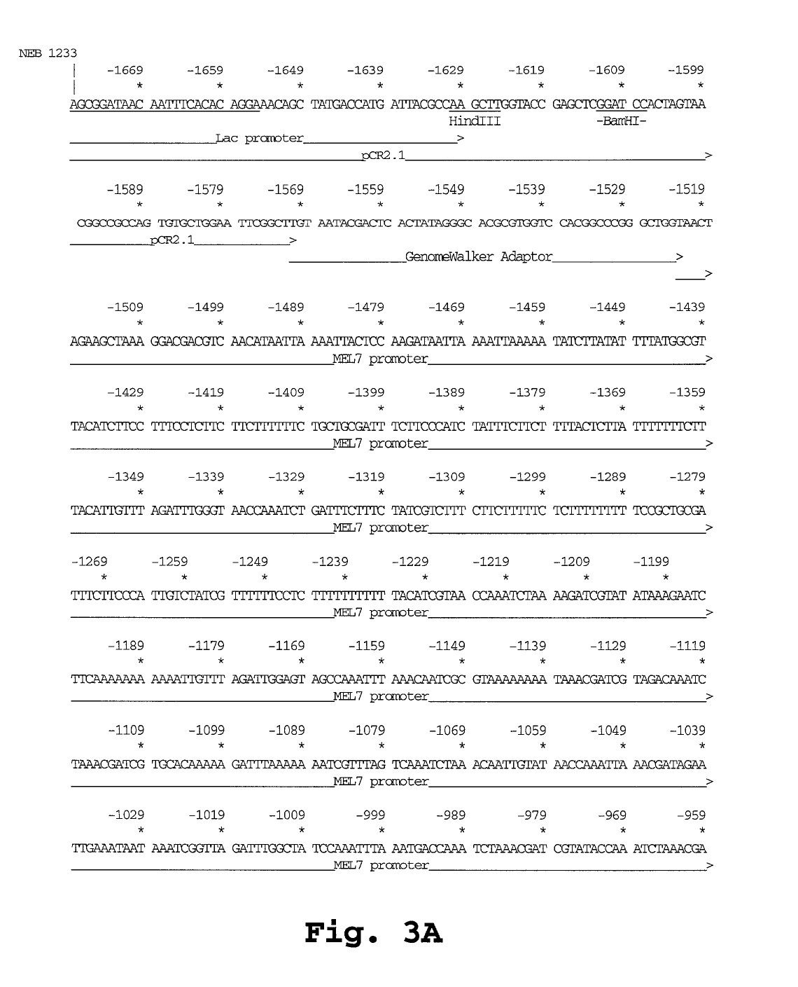

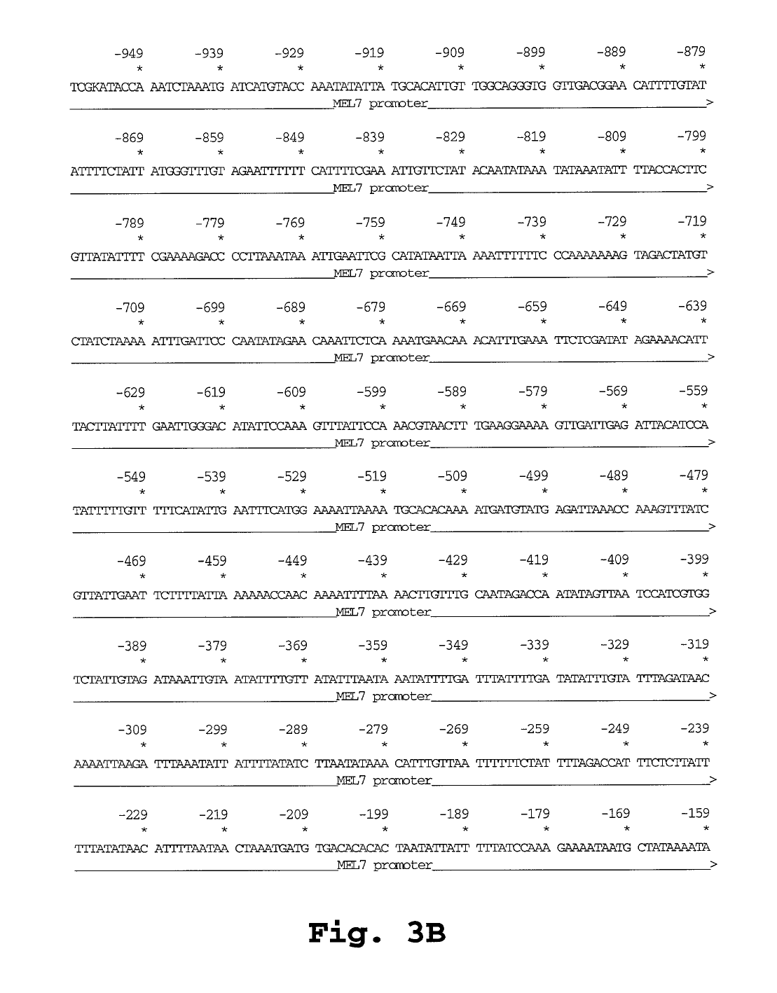

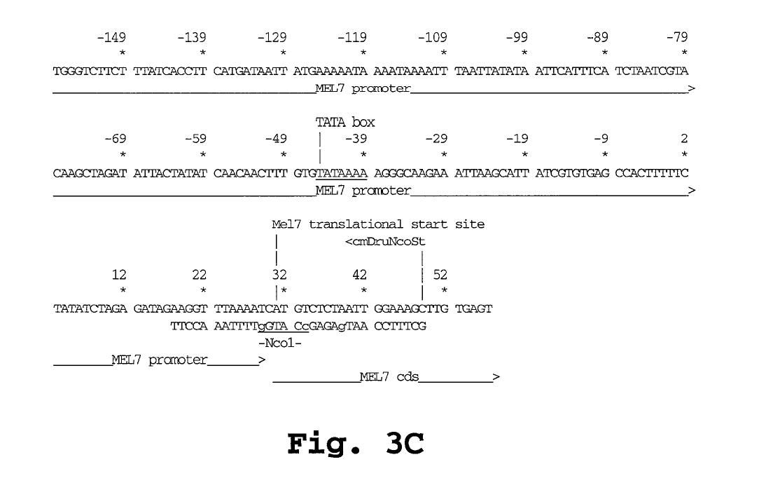

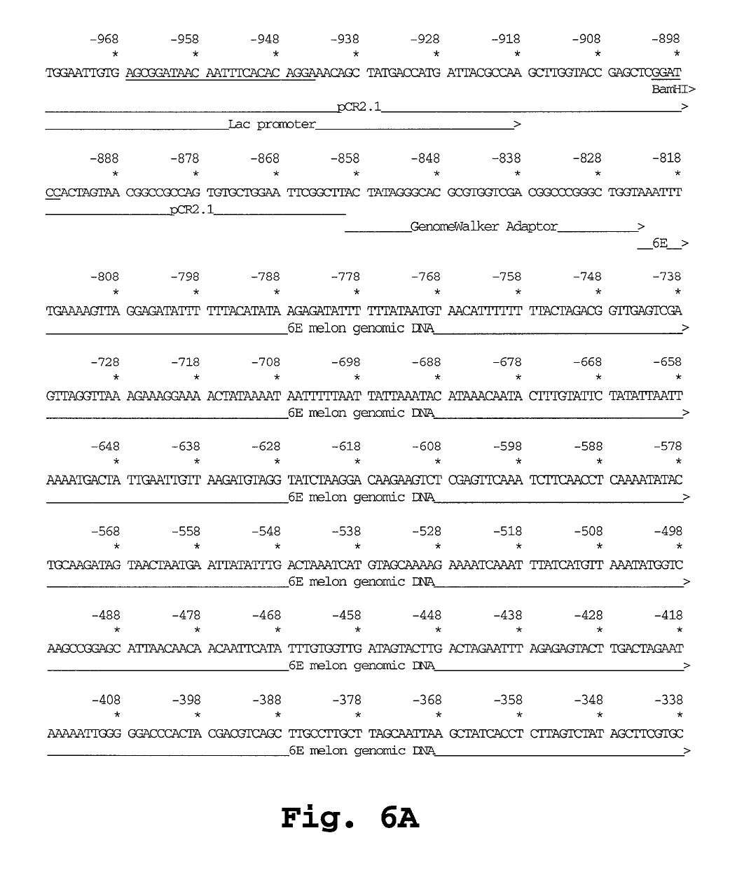

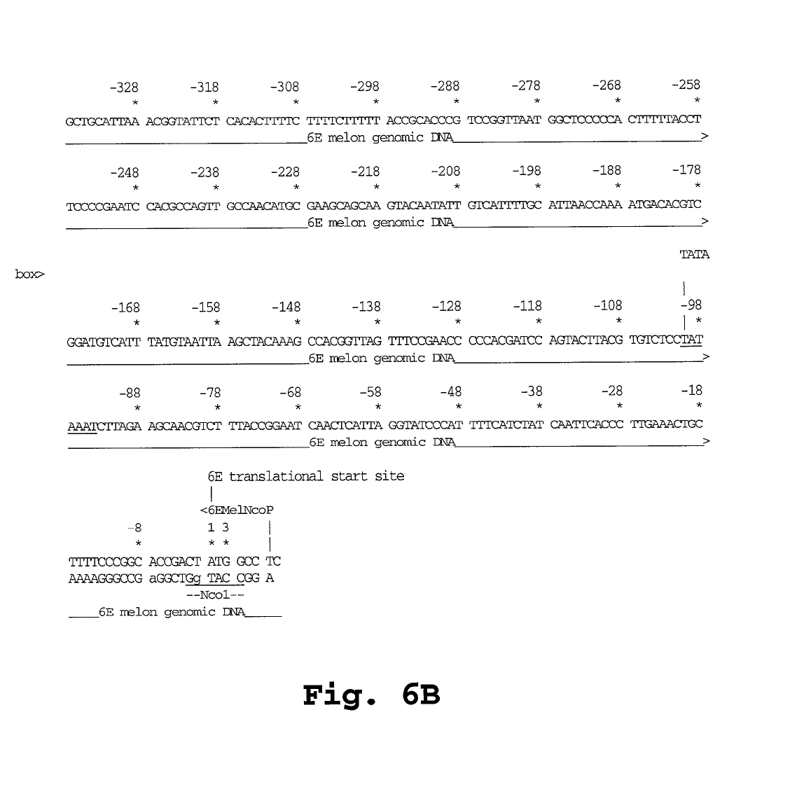

Sequences Provided in Support of the Invention
Unless otherwise indicated, all sequences are shown
in the conventional 5′ to 3′ orientation.

| | |
|---|---|
| | SEQ ID |
| DESCRIPTION | NO |

| | |
|---|---|
| Adaptor (universal genome walker): | 1 |
| 5′-GTAATACGACTCACTATAGGGCACGCGTGGTGGTCGACGGCCCGGGCTGGT-3′ | |
| Adaptor, complimentary strand: 3′-H2N-CCCGAGGA-PO4-5′ | 2 |
| AP1 primer: 5′-GTA ATA GGA GTG ACT ATA GGG C-3′ | 3 |
| AP2 primer: 5′-ACT ATA GGG CAC GCG TGG T-3′ | 4 |
| PFAcol#1 primer: 5′-AAT TTG CTC CAA TAT CTT AGC TCT AC-3′ | 5 |
| PFAcol#2 primer: 5′-AGA CAG CCA TTT CTT TTT GTA GAT AC-3′ | 6 |
| NEB #1233 primer: 5′-AGC GGA TAA GAA TTT CAC ACA GGA-3′ | 7 |
| ACOlProR-a primer 5′-TAGAGGGATGCTTCTTTTTGTAGATACAAGAT-3′ | 8 |
| E4UTR5′UP primer: 5′-GATCGATTATTAGAGATTGAGG-3′ | 9 |
| E4UTR5′LO primer: 3′-CATGGGTGAATCTCTAATAATG-5′ | 10 |
| cmDruP5′H3 primer: 5′-GGG CTG GAA AGC TTA AGA GAA ATT GGT A-3′ | it |
| cmDruP3Bam primer: 5′-GGG GTT TTG TTT TTG GAT CCT GGG TGT GTT-3′ | 12 |
| MAR AP1:5′-GCATCGTAATACGAGTCAGTATAGGGC-3′ | 13 |
| CSP: 5′-GGGCAGGTTTCTAGAATTCAGCGGCCGC-3′ | 14 |
| pM7-5R-1′ 5′-GTG AAA CTC GAC CCG TTC CTT AAA AAC TTC-3′ | 15 |
| cmDruNcoSt 5′-GCTTTCCAATGAGAGCCATGGTTTTAAACCTT-3′ | 16 |
| pMel2-outer: 5′-TAT TAG GTT GAG TGG ATG TGT TCG GTG-3′ | 17 |
| pMel2-inner: 5′-GCCTTAAGCTTTGTTGATCATCCACATC-3′ | 18 |
| MEL2 NcoR: 5′-GTT TGC ATT GTT TCC ATG GGA AA -3′ | i9 |
| NEB 1233: 5′-AGG GGA TAA CAA TTT CAC ACA GGA-3′ | 20 |
| H-T₁₁G: 5′-AAG CTT TTT TTT TTT G-3′(DD anchor primer, HindIII series) | 21 |
| H-T₁₁G: 5′-AAG CTT TTT TTT TTT C-3′(DD anchor primer, HindIII series) | 22 |
| H-T₁₁A: 5′-AAG CTT TTT TTT TTT A-3′(DD anchor primer, HindIII series) | 23 |
| H-AP1: 5′-AAG GTT GAT TGG G-3′(DD random primer, Hindlil series) | 24 |
| H-AP2: 5′-AAG CTT GGA GTG T-3′(DD random primer, Hindlil series) | 25 |
| H-AP3: 5′-AAG GTT TGG TGA G-3′(DD random primer, Hindill series) | 26 |
| H-AP4: 5′-AAG GTT CTG AAC G-3′(DD random primer, Hindlil series) | 27 |
| H-APS: 5′-AAG GTT AGT AGG G-3′(DD random primer, Hindill series) | 28 |
| H-AP6: 5′-AAG GTT GGA GGA T-3′(DD random primer, Hindill series) | 29 |
| H-AP7: 5′-AAG GTT AAG GAG G-3′(DD random primer, Hindill series) | 30 |
| H-AP8: 5′-AAG CTT TTA GGG G-3′(DD random primer, HindIll series) | 31 |
| 6E-PF1: 5′-TTG TAG GGG AAA AGG AAG TGG GCG TAA T-3′ | 32 |
| 6E-PF2: 5′-GGG AGA GTG ACG GGA AGA AAG AAT AGG-3′ | 33 |
| 6EMELNcoP: 5′-AGGGGATGGTGGGTGGGGGGAAAA-3′ | 34 |
| NEB #1233 (M13 Reverse, -40): 5′-AGG GGA TAA CAA TTT CAC ACA GGA-3′ | 35 |
| 2F-PF1: 5′-GA CAGTATAGTTCATGGCTTGGTTGG-3′ | 36 |
| 2F-PF2: 5′-AGGTTCTTTTAATCAGGCAATCTTCTT-3′ | 37 |
| 2FBamStart: 5′-GCGGGATCCTATTTTTGTGAATTGGAAATG-3′ | 38 |
| NEB #1224 (M13 Forward, -40): 5′-CGCCAGGGTTTTCCCAGTCACGAC-3′ | 39 |
| 1.35kb ACOl promoter (-1256 to the translational start at +101, FIGS. 1A-B) | 40 |
| ACOl/TE4 promoter (FIGS. 2A-B) | 41 |
| MEL7 promoter (FIGS. 3A-C) | 42 |
| MEL2 promoter (FIGS. 4A-C) | 43 |
| melon 6E promoter (FIGS. 6A-B) | 44 |
| melon 2F promoter (FIGS. 7A-G) | 45 |

What it is claimed:

1. An isolated nucleic acid sequence comprising a melon fruit-associated promoter selected from the group consisting of a cmACO1, cmACO1/TE4, MEL7, MEL2, 6E, and 2F promoter, characterized by the ability to promote fruit-associated expression of a transgene to which said melon fruit-associated promoter sequence is operably linked.

2. The melon fruit-associated promoter of claim 1 wherein the fruit-associated expression is ethylene regulated.

3. The melon fruit-associated promoter of claim 1 wherein expression is induced by changes in ethylene concentration in the plant and said melon fruit-associated promoter is activated, or primarily activated, during later stages of fruit development and/or early stages of fruit ripening.

4. The melon fruit-associated promoter of claim 1, wherein the melon fruit-associated promoter is a cmACO1/TE4 promoter having the nucleotide sequence presented as SEQ ID NO:41.

5. The melon fruit-associated promoter of claim 1, wherein the melon fruit-associated promoter is a MEL7 promoter having the nucleotide sequence presented as SEQ ID NO:42.

6. The melon fruit-associated promoter of claim 1, wherein the melon fruit-associated promoter is a MEL2 promoter having the nucleotide sequence presented as SEQ ID NO:43.

7. A plant expression vector comprising the promoter of any one of claims 4 to 6.

8. The plant expression vector of claim 7, operably linked to a heterologous nucleic acid coding sequence.

9. The plant expression vector of claim 8, operably linked to control sequences recognized by a host cell transformed with the vector.

10. The plant expression vector of claim 9, wherein said heterologous nucleie acid coding sequence encodes S-adenosylmethionine hydrolase (SAMase).

11. A plant cell comprising the plant expression vector of claim 9 or 10.

12. A mature plant comprising the plant cell of claim 11.

13. A plant cell comprising a melon fruit-associated promoter according to claim 1.

14. A mature plant comprising the plant cell of claim 13.

15. A method of expressing a heterologous nucleic acid sequence in a plant cell, comprising:
(a) transforming a plant cell with a nucleic acid construct comprising a melon promoter according to claim 1, operably linked to a heterologous nucleic acid coding sequence;
(b) culturing said plant cells in a culturing medium containing a selection agent to select for transformed plant cells.

16. The method according to claim 15, further comprising growing said transformed plant cells to produce a transgenic fruit-bearing plant.

17. The method according to claim 16, wherein said heterologous nucleic acid coding sequence is preferentially expressed in the fruit of said transgenic fruit-bearing plant.

18. The method according to claim 16, wherein the expression of said heterologous nucleic acid coding sequence is one or more of (a) ethylene regulated, (b) induced by changes in ethylene concentration in the plant, and (c) activated, or primarily activated, during later stages of fruit development and/or early stages of fruit ripening.

19. The method according to claim 18, wherein said heterologous nucleic acid coding sequence is sam-k and the mature fruit of said transgenic fruit-bearing plant exhibits a decrease in ethylene production relative to a non-transgenic plant.
